(19) United States
(12) Patent Application Publication
(10) Pub. No.: US 2017/0151286 A1
(43) Pub. Date: Jun. 1, 2017

(54) COMPOSITIONS AND METHODS FOR PREPARING AN INJECTABLE MEDIUM FOR ADMINISTRATION INTO THE CENTRAL NERVOUS SYSTEM

(71) Applicant: InVivo Therapeutics Corporation, Cambridge, MA (US)

(72) Inventors: Artem B. Kutikov, Somerville, MA (US); Richard T. Layer, Marlboro, MA (US); Simon W. Moore, Waltham, MA (US); Thomas R. Ulich, New York, NY (US); Alex A. Aimetti, Sudbury, MA (US)

(21) Appl. No.: 15/362,257

(22) Filed: Nov. 28, 2016

Related U.S. Application Data
(63) Continuation-in-part of application No. 15/361,985, filed on Nov. 28, 2016.
(60) Provisional application No. 62/384,505, filed on Sep. 7, 2016.
(60) Provisional application No. 62/261,622, filed on Dec. 1, 2015.

(51) Int. Cl. A61K 35/30; A61K 47/36; A61K 45/06; A61K 9/00

(52) U.S. Cl. CPC A61K 35/30; A61K 9/0085; A61K 9/0019; A61K 47/36; A61K 45/06

(57) ABSTRACT

Injectable mediums and methods for preparing and administering an injectable medium comprising therapeutic cells, and optionally one or more therapeutic or diagnostic substance, suitable for injection into an anatomical space of a human or animal subject, comprising hyaluronic acid in concentrations of 0.5 weight percent to 1.0 weight percent having a molecular weight of about ≧700 kDa to about 1,900 kDa and a storage modulus within the range of 5-25 Pa, which injectable mediums and methods prevent cell settling during transportation and storage of such injectable mediums comprising therapeutic cells, and optionally therapeutic or diagnostic substances; promote cell survival; facilitate administration of homogeneous injectable mediums comprising therapeutic cells, in particular NSCs; and enable rapid clearance by the body following injection, so as not to interfere with cellular integration with surrounding tissue.

### CROSS-REFERENCE TO RELATED APPLICATION(S)

This application claims the benefit of U.S. Provisional Application No. 62/261,622, filed on Dec. 1, 2015 and U.S. Provisional Application No. 62/384,505, filed on Sep. 7, 2016, and also claims priority to non-provisional patent application Ser. No. 15/361,985 entitled METHODS AND SYSTEMS FOR DELIVERY OF A TRAIL OF A THERAPEUTIC SUBSTANCE INTO AN ANATOMICAL SPACE filed on the same date as the present application. The entire disclosure of each of the aforesaid applications is incorporated by reference in the present application.

### BACKGROUND OF THE INVENTION

Field of the Invention

The present invention is directed to injectable and biodegradable compositions comprising hyaluronic acid and therapeutic cells, and optionally therapeutic or diagnostic substances, and to methods of making and administering such compositions for the purposes of delivering and administering therapeutic cells and/or therapeutic or diagnostic substances to diseased or injured anatomical spaces of the body of an animal or human subject, preferably to the central nervous system (“CNS”), and, in particular, to a diseased or injured spinal cord, of an animal or human subject.

Description of Related Art

Cell therapy refers to the transfer of cells into a human or animal subject with the goal of improving a disease, condition, or injury to such subject. In particular, cell therapy also refers to the administration to a human or animal subject autologous, allogeneic, or xenogeneic living non-germline cells for therapeutic, diagnostic, or preventive purposes. Cell therapy compositions may also comprise therapeutic cells which have been manipulated or processed ex vivo. Cells may also be modified ex vivo for subsequent administration to humans or animals, or may be altered in vivo by gene therapy administered directly to the subject. When the genetic manipulation is performed ex vivo on cells which are then administered to the patient, this is also considered to be a form of cell therapy.

Gene therapy involves a medical intervention that transiently or permanently modifies the genetic material of living cells. The modification may involve adding, subtracting or replacing genetic information. The genetic manipulation may be intended to have a therapeutic or prophylactic effect in the subject receiving gene therapy. Vectors (e.g. viruses, liposomes), additives (e.g. polybrene), recombinant RNA or DNA materials used to modify the genetic material of cells are considered components of gene therapy. The genetic material may encode a product or products (e.g., enzyme, protein, polypeptide, peptide, non-coding RNA, coding RNA) or regulate the expression of a gene product (e.g. enhance or repress). The gene product may encode a hormone, receptor, enzyme, polypeptide, peptide, interfering RNA, targeted gene editing products (e.g. meganucleases, zinc finger nucleases (ZFNs), transcription activator-like effector-based nucleases (TALEN) or CRISPR-Cas9) of therapeutic value. For a review see “Gene and Cell Therapy: Therapeutic Mechanisms and Strategies, Fourth Edition” Nancy Smyth Templeton (2015).

Most cell therapy treatments and compositions, as well as gene therapy treatments, are regulated in the United States by the Food and Drug Administration (“FDA”). Similar regulatory oversight of cell and gene therapies exists through other regulatory agencies on a global basis. Cells for therapeutic purposes (also referred to as “therapeutic cells,” as identified below, may be formulated and delivered in a variety of manners, including by infusion, injection at various sites, or they may be surgically implanted in aggregated form or along with solid supports or encapsulating materials. The FDA categorizes any such matrices, fibers, beads, or other materials which are used in addition to the cells as excipients, additional active components or medical devices.

Therapeutic or diagnostic applications may be proposed involving cells which are modified by recombinantly modifying the cells to produce a bioactive substance or by pre-loading with bioactive materials. Accordingly, the cell implant may be used as a delivery system not only for its own products and functions but also for other products.

Therapeutic cell therapy may be employed to repair, replace or restore damaged tissues in anatomical spaces of a human or an animal subject, for example tissue within the CNS, more particularly, tissue within the spinal cord. Cell therapy is still a nascent therapeutic approach to treating human diseases, conditions and injuries, in particular CNS injuries, and especially spinal cord injuries due to disease or injury (“SCI”(s)).

The U.S. FDA has approved a cell therapy product, HEMACORD® Injectable suspension (HPC, Cord Blood) as the first cell therapy product for use in humans. HEMACORD comprises an allogeneic cord blood hematopoietic progenitor cell therapy composition indicated for use in unrelated donor hematopoietic progenitor cell transplantation procedures in conjunction with an appropriate preparative regimen for hematopoietic and immunologic reconstitution in patients with disorders affecting the hematopoietic system that are inherited, acquired, or result from myeloablative treatment. The product is administered as an intravenous infusion and the composition comprises hematopoietic progenitor cells which express the cell surface marker CD34. The potency of cord blood is determined by measuring the numbers of total nucleated cells (TNC) and CD34+ cells, and cell viability and certain inactive pharmaceutically acceptable excipients. Each unit of HEMACORD contains a minimum of 5×10⁸ total nucleated cells with at least 1.25×10⁶ viable CD34+ cells at the time of cryopreservation. The inactive pharmaceutically acceptable excipients consist of dimethyl sulfoxide (DMSO) and Dextran 40.

Spinal cord injuries may result in paraplegia or quadriplegia in a substantial number of subjects. Over 250,000 people annually in the U.S. alone are believed to suffer spinal cord injuries due to trauma. The delivery of therapeutic cells, and optionally therapeutic or diagnostic substances, such as growth factors, antibodies, analgesics, anesthetics and the like, into the spinal cord, may be useful in the treatment of spinal cord injuries, amyotrophic lateral sclerosis (“ALS”), multiple sclerosis, spinal muscular atrophy, and spinal ischemia as well as other spinal cord degenerative conditions and pathologies.

Prior delivery strategies for the injection of a therapeutic substance into the central nervous system have a number of limitations. Some injection strategies require multiple injection sites, thereby resulting in concentrated and localized delivery sites for cells and/or other therapeutic substances. For instance, in one procedure multiple vertical spinal cord injections are required to deliver cells into multiple spinal cord segments. Such a procedure presents risks to the patient, such as infection and loss of cerebrospinal fluid and the attendant sequelae due to the multiple injections required.

Injections of the type described above may, for example, cause injury at each site of injection; deliver inaccurate doses as a result of cell reflux up the needle track; have limited surface area for cellular integration, or require lengthy procedure times.

Delivery of cells and/or therapeutic substances directly into the parenchyma of the spinal cord thus presents numerous challenges to a health care professional. These challenges include the relatively small size of the spinal cord, movement of the spinal cord within multiple planes relative to the surrounding vertebrae, and the known vulnerability of the spinal cord to injury. Therefore, a need exists to provide a minimally invasive technique and associated apparatus for the administration of therapeutic cells, and optionally therapeutic or diagnostic substances, directly into the traumatized and/or diseased central nervous system, particularly the spinal cord

Systems known in the art for administration of therapeutic cells, and optionally therapeutic or diagnostic substances, into the central nervous system include injections into the brain using multiple injections of cells through flexible, plastic cannulas. The multiple injections result in localized deposition of cells that are not in a single plane. See FIG. 2C of Brecknell and Fawcett, Experimental Neurology, 1996; 138: 338-343. Another administration device employs a rigid guide needle which maintains a specific angle for placement of a flexible injection needle. See page 1498—Material and Methods section of Cunningham et al., Neurosurgery, 2004; 54: 1497-1507.

The procedures and apparatus described in the foregoing references depart significantly from the procedures and apparatus described in U.S. Provisional Application No. 62/261,622, filed on Dec. 1, 2015 and U.S. Provisional Application No. 62/384,505, filed on Sep. 7, 2016. Prior injection methods differ, for example, by utilizing a non-motorized flexible cannula as opposed to a motorized injection cannula housed in a guide needle assembly and the lack of control over injection angles that is evident in the prior disclosures. The procedures and apparatus disclosed in the foregoing references would be unsuitable to deliver a long trail of therapeutic cells, and optionally therapeutic or diagnostic substances, into the narrow diameter (generally on the order of <1 to 1.5 cm) of the spinal cord. The invention described in U.S. Provisional Application No. 62/261,622, filed on Dec. 1, 2015 and U.S. Provisional Application No. 62/384,505, filed on Sep. 7, 2016 as well as an application filed of even date herewith entitled “Methods and Systems for Delivery of a Trail of a Therapeutic Substance Into an Anatomical Space,” the entire contents of which are hereby incorporated by reference, solves these and other administration problems by controlling penetration of the injection needle at a relatively shallow angle, generally on the order of 5-25°. Moreover, it is not feasible to implement the prior art cranial injection devices and injection procedures for delivery of therapeutic cells, and optionally therapeutic or diagnostic substances, into the spinal cord because the cranial injection cannulas cannot be deflected through a side hole aperture at such an angle, as disclosed in the references.

Another injection system employs an endoscope comprising a large (10 gauge) needle attached at the distal end and a flexible, steerable endoscope housing a microcatheter, which may be directed intradurally through an introducer sheath. Saline is introduced to distend the subarachnoid space via a syringe in communication with the needle affixed to the endoscope. The microcatheter with attached needle is advanced through a working channel of the endoscope into the dorsal surface of the spinal cord. Therapeutic cells may then be introduced while the microcatheter is withdrawn slowly to create a trail of therapeutic cells within the spinal cord. The spinal cord can be visualized through the skin puncture and the endoscope can be navigated under visual guidance. Therapeutic cells and/or a therapeutic substance(s) can be injected from the needle into the spinal cord. One such system is described in U.S. Pat. No. 7,666,177, the disclosures of which are hereby incorporated by reference in their entirety

In a particular embodiment, U.S. Pat. No. 7,666,177 (hereinafter, “the '177 patent”), the entire contents of which are hereby incorporated by reference herein, describes a procedure and system that includes injecting a therapeutic substance from a hollow guidewire and withdrawing the guidewire over a period of time to create a trail of therapeutic substance parallel to the longitudinal axis of the spinal cord. Such a system requires use of an endoscope and injection of a fluid to distend the epidural and subarachnoid spaces of the spinal cord, thereby complicating the administration of therapeutic cells and/or other therapeutic substances

The '177 patent thus employs an endoscope to access the subarachnoid space of the spinal cord and to introduce a needle to deliver a trail of therapeutic parallel to a longitudinal axis of the cord. Such a procedure is described in Guest, J. et al., Neurosurgery 54(4): 950-955 (3004). Table 1 of the reference publication notes various problems associated with this endoscopic injection approach versus open surgical approaches. These problems include: potential alteration of the subarachnoid space after injury may render the approach unfeasible; visualization through an endoscope is typically poor compared to a surgical microscope; the trajectory of the injection needle may be constrained; and cellular dispersion may be increased in a fluid environment, resulting in seeding outside the desired injection site.

The described endoscope-based approach in the '177 patent and the foregoing publication is technically challenging due to the limited spinal cord access and visualization provided by an endoscope. This approach also lacks reproducibility because the trail of therapeutic cannot be stereotaxically positioned within the cord, and the injection procedure and described injection apparatus lacks control of trail length and volume due to the described manual approach. The foregoing technical challenges and lack of accuracy may result in the creation of short trails of cells and/or therapeutic substances of only 4-mm in length, as described in the specification. Such a distance is insufficient to bridge most spinal cord lesions. Finally, trails created at an angle with respect to the cord, rather than parallel to the cord axis, may be therapeutically beneficial and these cannot be accomplished with the apparatus and injection procedure specified in the '177 patent

Another injection system known in the art is described in U.S. Pat. No. 9,011,410 (hereinafter, “the '410 patent”), which is incorporated herein by reference in its entirety. The '410 patent describes a drug or cell delivery system for multi-segmental injection of therapeutic cells, and optionally therapeutic or diagnostic substances, into the spinal cord of an animal or a human. The device provides for delivery of a substrate into a spinal cord and comprises a guide needle having an inside diameter; an injection needle fitting into the inside diameter of the guide needle; a stepping motor advancing the injection needle into and within the spinal cord; and a chamber containing the substrate or cells in fluid communication with the injection needle. In operation, the device may deliver a substrate into a spinal cord. The administration method comprises advancing a guide needle into the spinal cord, the guide needle having a bend at an angle of about 45 degrees at an end thereof, the end being advanced into the spinal cord; advancing an injection needle through the guide needle and into the spinal cord with a stepping motor attached to the injection needle; and then injecting the substrate into the spinal cord through a syringe attached to the injection needle. The external end of the injection needle is directly connected to the syringe with polyethylene tubing. When the injection needle is withdrawn, the cells and/or other therapeutic substance may be injected into the spinal cord. The stepping motor attached to the injection needle between the syringe and a portion of the injection needle inside the guide needle may provide for multi-segmental delivery of the cells and/or other therapeutic substance into the spinal cord. The foregoing apparatus requires a fixed bend to the guide needle into the spinal cord that in certain instances impedes the positioning of the injection needle within the spinal cord and therefore may impair the deposition of a longitudinal trail of cells and/or other therapeutic substances within the spinal cord parenchyma.

As discussed above, the '410 patent discloses a device and method for multi-segmental delivery of a substrate into the spinal cord employing a bent guide needle, stepper motor controlled injection needle, and syringe. An important component of the '410 patent is the described guide needle and associated method. This guide needle has a 45 degree bend at the tip and is inserted at a 45 degree angle in relation to the cord. Inserting such a needle into the spinal cord might cause substantial damage to the spinal cord. Furthermore, there is no way to control the angle of the resulting therapeutic trail. Differing patient anatomies, pathologies, and therapeutic mechanisms may require alternate angles of trails within the spinal cord. The ability to create two trails that meet at a vertex, like a tent, may also be of therapeutic benefit and is not possible with the device\method described in '410 patent. Moreover, the described device states that a stepping motor is attached to the injection needle between the syringe and a portion of the injection needle. This arrangement alone does not enable insertion of an injection needle into the spinal cord because the injection needle may buckle between the guide needle and stepper motor attachment. Another disadvantage of the disclosed method is the polyethylene tubing used to connect the injection needle and the syringe. This flexible polyethylene tubing increases the dead volume between the syringe and the tip of the injection needle, potentially resulting in loss of therapeutic and reduced control of the injection flow rate and delivery volume.

The injection device and methods of the inventions described in in U.S. Provisional Application No. 62/261,622, filed on Dec. 1, 2015 and U.S. Provisional Application No. 62/384,505, filed on Sep. 7, 2016, as well as an application filed of even date herewith entitled “Methods and Systems for Delivery of a Trail of a Therapeutic Substance Into an Anatomical Space, are capable of depositing trails of therapeutic cells, and optionally therapeutic or diagnostic substances that may cross an injury of the spinal cord between two points along the longitudinal axis of the spinal cord. The two points may be rostral and caudal to an injury site of the cord and may be due to a compression or contusion injury or the severance or partial severance of the spinal cord. For conditions such as ALS, the trail may not cross an injury site per se, but rather the injection of a trail of therapeutic cells, and optionally therapeutic or diagnostic substances may enable the continuous application of cells into a diseased cord without multiple puncture sites. For instance, in treating ALS, the trail of cells may be positioned near the ventral horn motor neurons. The same would be true with respect to MS, where remyelination of the axons of diseased spinal cord neurons may be an objective of the injection of trails of therapeutic cells, and optionally therapeutic or diagnostic substances. The same would also hold true for other ischemic and pathological conditions of the spinal cord. In the foregoing treatments, one of the principal objectives, therefore, is to minimize the number of penetrations into the spinal cord parenchyma.

The development of cell therapies is the focus of investigations for the treatment of numerous indications with currently unmet needs. Such therapies administered as a cell suspension ideally require the use of a vehicle that is compatible with the cells, non-toxic to the recipient, does not compromise the post-transplantation cellular environment, and is suitable for storage of the therapy for a sufficient time prior to and during administration.

It is known in the art that neural stem cells are capable of replacing cells in damaged and/or degenerating CNS neural pathways through cell replacement therapies (also referred to as “cell therapies”). Mice treated with 1-methyl-4-phenyl-1,2,3,6-tetrahydropyridine (MPTP) to selectively destroy dopaminergic cells in the brain stem, that were subsequently transplanted with a neural stem cell population, regenerated a dopaminergic cell population composed of both donor and host cells. In a second study in mice using a hypoxia-ischemic brain injury model. The transplantation of neural stem cells enhanced the recovery of the damaged nervous system (Park et al. (1999) J. Neurotrauma 16:675-687 and Park et al. (1997) Soc. Neurosci. Abst. 23:346).

In stroke patients, the transplantation of cells from a human neural cell line showed improvement of neurological function. (Kondziolka D., et al., (2000) “Transplantation of cultured human neuronal cells for patients with stroke”. Neurology. 55:565-9).

Similarly, in a mouse model of Alzheimer's disease, the transplantation of neural stem cells into the prefrontal and parietal cortices dramatically alleviated the cholinergic deficits and recent memory disruption associated with AD. (Wang, Q., et al., (2006) “Neural stem cells transplantation in cortex in a mouse model of Alzheimer's disease. J Med Invest., 53:61-9).

Further studies have been performed in Parkinson's disease where it has been shown that stem cell transplants of different origin, e.g., hematopoietic, embryonic, result in several clinical benefits in patients with severe Parkinson's disease. (Freed, C. R., et al., Transplantation of embryonic dopamine neurons for severe Parkinson's disease, New Engl. J. Med. 2001; 344:710-719). Multiple sclerosis patients have shown improvements with stem cell therapies. Ni, X. S., et al., (2006), “Autologous hematopoietic stem cell transplantation for progressive multiple sclerosis: report of efficacy and safety at three year of follow up in 21 patients” Clin. Transplant. 20:485-9.

Similarly a study in Huntington Disease suggests the benefit of human fetus-to-adult striatal cell transplantation. Kopyov et al. (1998) J. Exp. Neurol. 149:97-108.

Benefits of stem cell therapeutic transplantation in spinal cord injuries has also been shown in the implantation of polymeric multicomponent PLGA scaffolds seeded with neural stem cells were implanted into SCI rat hemi-section model demonstrated new tissue containing neurons or neuronal elements. Teng, et al. PNAS 99(5):3024-3029. Transplanted neural stem cells have been shown to extend long processes, form synaptic connections with host neurons in the spinal cord, and result in functional recovery from transection injury in rats. Lu et al., Long-Distance Growth and Connectivity of Neural Stem Cells after Severe Spinal Cord Injury. Cell, 2012 Sep. 14; 150(6): 1264-1273.

Although the use of hyaluronic acid-containing compositions alone or in combination with methylcellulose and other excipients and human neural stem cells has been known and cited in the art, a substantial need still exists for compositions which do not pose significant biocompatibility issues and risks to a subject, including a human or an animal, upon injection into the central nervous system, for example, injection into the spinal cord parenchyma and/or surrounding tissues. Examples of compositions which may pose potential biocompatability issues are liquid compositions comprising, methylcellulose. A number of compositions known in the art, which may comprise human neural stem cells, may become difficult to inject because of increased viscosities when exposed to room temperature after being stored on ice or at cool temperatures for transportation or for other storage reasons.

A result of cellular aggregation, disruption and sedimentation of neural stem cells in liquid suspension is that the delivery of uniform cell suspensions to the spinal cord cannot be achieved. This problem may occur in numerous settings, for instance, in vials containing sterile suspensions of neural stem cells manufactured for various therapeutic indications. Alternatively, cellular aggregation, disruption and sedimentation may occur in pre-loaded syringes comprising suspensions of stem cells.

Another example of the problem caused by cellular aggregation, disruption or sedimentation may occur in cell injection and delivery systems such as those described in U.S. Pat. No. 7,666,177, which describes a method of injecting a therapeutic substance comprising cells into the spinal cord of a human subject. Another application is in a spinal multisegmental cell and drug delivery system such as described in U.S. Pat. No. 9,011,410.

Another application is in transplantation of a trail of neural stem cells into the spinal cord parenchyma using, for example, the injection devices of U.S. Provisional Application Nos. 62/261,622, filed Dec. 1, 2015; and U.S. Provisional Application No. 62/384,505, filed Sep. 7, 2016, as well as an application filed of even date herewith entitled “Methods and Systems for Delivery of a Trail of a Therapeutic Substance Into an Anatomical Space,” which applications describing various injection delivery systems are incorporated by reference herein.

It is understood in the art that stem cells of various types may aggregate in liquid compositions, particularly upon isolation of the stem cells from biological specimens. Consequently, intact tissue fragments have been subjected to disaggregation procedures using a variety of conventional methods such as mechanical force, enzymatic digestion with single or combinatorial proteolytic enzymes, such as collagenase, trypsin, lipase, liberase H1, as disclosed in U.S. Pat. No. 5,952,215, and pepsin, or a combination of mechanical and enzymatic methods.

Liquid compositions for the prevention of cellular aggregation in suspensions of stem cells have been described in the art. A composition comprising aspirin or aspirin lysine and further comprising Hartman-D solution or phosphate buffered saline (PBS) is described in US Patent Publication No. 2015-0118194. However, long-term storage effects of storing stem cell suspensions in aspirin containing compositions is not described nor is the impact of such aspirin-containing composition on the viability of stem cells delivered therapeutically to a human subject described, or the toxicity of injecting such compositions into the spinal cord parenchymal tissue.

U.S. Patent Publication No. 2007-0048288 describes various shear thinning polymer cell delivery compositions for delivery of a variety of cells, including stem cells, e.g., pluripotent stem cells, mesenchymal stem cells, endothermal stem cells, ectodermal stem cells employing shear thinning high molecular weight poly(alkylene oxide) polymers. The shear thinning polymers are stated to reduce cell settling and facilitate the delivery of cells when placed in a carrier liquid at an appropriate concentration. Hyaluronic acid is not disclosed as such a shear-thinning polymer.

Methods for inhibiting stem cell aggregation in liquid compositions are also taught in U.S. Patent Publication No. 2012-0020931 using a soluble inducer retinoic acid in compositions comprising stem cells encapsulated using an alginate polyelectrolyte microenvironment. In the presence of retinoic acid supplementation, encapsulated cells showed decreased cell aggregation compared to non-supplemented cells. Encapsulated cells in the presence of retinoic acid remained as single cells throughout the 20 days culture period. In contrast, differentiating cells cultured in the basal medium configuration continued to aggregate. Hyaluronic acid is not mentioned as a component in the described liquid compositions.

U.S. Patent Publication No. 2011-0208162 describes a mixture of a population of adipose-derived stromal cells (ASC) and an ASC aggregation inhibitor. ASC aggregation inhibitors disclosed are heparin, an integrin inhibitor, EDTA, trypsin and dispase. Hyaluronic acid is not mentioned among the described ASC aggregation inhibitors.

U.S. Patent Publication No. 2015-0010501 discloses preparations and methods for treating intervertebral disc injuries in a mammal having discogenic pain comprising a formulation of ELA stem cells lacking expression of certain detectible cell surface markers in combination with a hyaluron, for example hyaluronic acid (HA). The compositions described are silent with regard to aggregation of the stem cells and does not disclose neural stem cells.

U.S. Patent Publication No. 2015-0202295 discloses certain shear-thinning and stabilizing hydrogel compositions comprising a hydrophilic polymer network. The hydrophilic polymer network comprises non-covalent crosslinks and at least one set of chemical moieties being capable of participating in at least one chemical covalent cross-linking reaction. The hydrophilic polymer network may comprise hyaluronic acid as a first or first and second hydrophilic polymer. Various types of stem cells are identified, including adipose derived stem cells, embryonic stem cells, bone marrow stem cells, cord blood stem cells, mesenchymal stem cells, adult stem cells, and pluripotent or induced pluripotent stem cells. Mesenchymal stem cells are preferred. Prevention of stem cell aggregation in such hydrogel compositions is not disclosed.

Culturing of stem cell aggregates, for example mesenchymal stem cells, in 3-dimensional matrices comprising among other polymers, hyaluronic acid, is disclosed in U.S. patent Publication No. 2015-0259648. In such compositions stem cell aggregates, as opposed to non-aggregation of stem cells, is the objective.

U.S. Pat. No. 6,290,729 describes certain thixotropic and pseudoplastic polymers, which exhibit shear thinning, whereby the polymer becomes more fluid under shear, and then reverts to a high-viscosity or gelled form on cessation of shear. An example of a material altering viscosity from a liquid to a gel upon exposure to shear or other physical forces is hyaluronic acid (HA), most preferably of a high molecular weight in the range of 300,000 daltons or more, at concentrations of about 1% or more. The HA can also be crosslinked ionically. The reduction of cellular aggregates in liquid compositions for delivery to an injury site is not disclosed.

U.S. Patent Publication No. 2011-0059182 describes a method of delivering multicellular aggregates to a target surface of a subject via a spraying device. The target surface may comprise at least one of a wound, tissue, or organ. The described method involves obtaining multicellular aggregates (MA); suspending the multicellular aggregates (MA); and spraying the suspension of multicellular aggregates (MA) on the target surface. Cells delivered in this manner may include: Human adipose stem cells (HASCs), human adipose progenitor cells (HAPCs), human adipose endothelial (HAECs), and human adipose stromal cells (HAStrCs) or any combination thereof. The suspension comprises a fluid or medium which may comprise at least one of the following: biologic and synthetic biocompatible systems such as reverse-thermal gelling poloxamers (e.g. Pluronic F68, Pluronic F127), chitosan, hyaluronic acid, hydrogels, buffers, saline, thrombin/fibrin, or platelet rich plasma (PRP), etc. or any combinations thereof. The reference however does not specify formulations of the cells and hyaluronic acid or that the liquid compositions prevent aggregation of the cells during administration.

U.S. Patent Publication No. 2007-0292401 discloses certain compositions comprising stem cells, including neural stem cells, which may employ a pharmaceutically acceptable carrier that may comprise a biodegradable substance. Examples include of such a carrier include hyaluronic acid and saline; however, the reference is silent as to prevention of aggregation of the cells during administration.

U.S. Pat. No. 8,980,248 describes injectable polymer compositions for use as cell delivery vehicles. The compositions contain a thermal gelling polymer, an anionic gelling polymer and a water-based carrier, which is injectable due to the shear thinning properties of the composition, wherein the viscosity and polymer network density are effective to promote cell survival in vivo and in vitro and which maintains a distribution of cells after injection. The thermal gelling polymer may be methylcellulose and the anionic gelling polymer may be hyaluronic acid. The compositions disclosed are said to be shear thinning compositions which permit cells to be mixed at room temperature but which maintains even distribution of cells after injection. Experiments using compositions employing both methylcellulose and hyaluronic acid for delivery of neural stem/progenitor cells into the spinal cord of rats are described for the purpose of determining whether the delivery vehicle promoted cell survival following transplantation. The Examples all relate to the cell suspensions employing HAMC polymers and not to liquid composition employing hyaluronic acid alone. The beneficial cell transplantation properties of the liquid compositions is based upon the unique combination and ratios of HA and MC.

U.S. Patent Publication No. 2004-0191225 describes an injection system for injecting cells, including stem cells and neuronal cells, using a side port needle, for example a spinal needle for anesthesia and lumbar puncture, and a liquid composition comprising a carrier which may be methylcellulose, hyaluronic acid, agarose or alginic acid. The composition may be injected into various organs of the body, including the spinal cord. The concept of cellular aggregation and sedimentation of liquid suspensions of stem and/or neural cells is neither disclosed nor contemplated in the reference. The Examples relate to injection of myoblasts into the hearts of pigs, sheep and humans in connection with coronary artery bypass surgery. The reference fails to disclose specific liquid compositions of suspended cells other than to note that the cells are provided as a homogeneous suspension in medium or some other solution, for example an isotonic or hypertonic solution.

U.S. Patent Publication No. 2015-0064143 discloses certain ionically cross-linkable alginate-grafted hyaluronate compound containing alginate and hyaluronate, the alginate and the hyaluronate forming a covalent linkage. The cross-linked hyaluronate polymers are disclosed as useful for cell transplantation procedures, but the reference is silent as to transplantation into the spinal cord. In addition, the reference fails to disclose specific formulations of the cells and hyaluronic acid alone or that the liquid compositions prevent aggregation of the cells during administration.

Hyaluronic acid containing hydrogels are disclosed in WO/2016/025945 for stem cell transplantation, but the reference does not disclose transplantation into the spinal cord, nor does the reference disclose specific formulations of the cells and hyaluronic acid alone or that the liquid compositions prevent aggregation of the cells during administration.

U.S. Patent Publication No. 2015/037474 A1 discloses certain methods and compositions for administration of cell therapy, comprising the steps of, administering a therapeutically-effective amount of therapeutic cells to the treatment site; and administering an effective amount of a biodegradable material comprising a hyaluronan compound to the treatment site, wherein the biodegradable material has an in vivo degradation profile similar to that of a hyaluronic acid having a molecular weight of 20 kDa to 2,000 kDa. The methods focus upon maintaining a population of therapeutic cells administered to a treatment site in a subject for a period of time. Hyaluronan compositions may be up to about 5% by weight of the composition and therapeutic cells may be present in concentrations of 1×10⁴ to about 1×10⁸ cells in a single dosage volume of about 100 to about 2,000 μL are described. While discussing slowing down the removal of cells in vivo following administration of the hyaluronan-stem cell compositions, the reference is silent as to preventing cellular aggregation and minimizing the disruption and sedimentation of stem cells during transport, storage and administration of such liquid compositions. Moreover, the examples of the reference do not relate to neural stem cells.

### SUMMARY

The present invention is based upon the discovery of injectable mediums for therapeutic cells, and optionally therapeutic or diagnostic substances, in particular neural stem cells, and hyaluronic acid, which prevent cell settling during transportation and storage of such injectable mediums of therapeutic cells, and optionally therapeutic or diagnostic substances, promote cell survival, facilitate administration of homogeneous therapeutic cell suspensions, in particular homogenous NSC suspensions, and enable rapid clearance by the body following injection so as not to interfere with cellular integration with surrounding tissue.

Importantly, combining the therapeutic cells with the carrier should not adversely affect cell viability during mixing, or upon injection or at the transplantation site.

Sedimentation of therapeutic cells, such as neural stem cells, due to cellular aggregation may occur in storage solutions for therapeutic cells, therapeutic cell delivery systems and therapeutic cell delivery compositions, as described previously herein. Moreover, the sedimentation of cells may occur almost instantaneously after injection, with the cells rapidly advancing down an angled injection trail to be deposited in an undesirable mass.

The present invention enables the preparation of storage stable liquid mediums of suspended therapeutic cells, and optionally therapeutic or diagnostic substances, for the manufacture, storage and delivery of therapeutic cells to a target delivery site, i.e. an anatomical space within the body of a human or an animal subject, particularly in the CNS, and especially the spinal cord, of a subject, in various diagnostic and therapeutic settings.

Injectable compositions comprising hyaluronic acid (“HA”) and therapeutic cells, and optionally therapeutic or diagnostic substances are useful in applications where there exists a need for delivery of uniform suspensions comprising hyaluronic acid, and viable populations of therapeutic cells, and optionally therapeutic or diagnostic substances, for purposes of cell transplantation and cell therapy into a site of injury within an anatomical space of the body, in particular injections into the central nervous system (“CNS”) including the brain and, most preferably, injections directly into the spinal cord. Such compositions provide for delivery of viable populations of therapeutic cells, and optionally therapeutic or diagnostic substances to enhance the survival, differentiation and integration of transplanted cells into the body, including the CNS and the spinal cord of a human or animal subject.

Cell delivery and the subsequent survival of transplanted cells are significant problems to be solved to provide for successful cellular transplantation. Most transplanted cells frequently die or migrate away from the transplant site and/or aggregate together. The result is that transplanted cells may not integrate with the host tissue. Klassen, H. J., Ng, T. F., Kurimoto, Y., Kirov, I., Shatos, M., Coffey, P. et al., “Multipotent retinal progenitors express developmental markers, differentiate into retinal neurons, and preserve light-mediated behavior,” Invest Ophthalmol Vis. Sci., 45(11):4167-73 (2004); Potts, M. B., et al., “Devices for cell transplantation into the central nervous system: Design considerations and emerging technologies,” Surg. Neurol Int., 4(Suppl) S-22-S30 (2013).

Successful implementation of cellular therapy requires cell survival, appropriate cell distribution, and implanted cell integration with tissue. Furthermore, translational concerns such as stability during transportation and administration must be addressed. When examining the delivery of cells such as neural stem cells (NSCs) for treatment of pathologies such as spinal cord injury, the cells should be delivered in a minimally invasive fashion (injectable) and differentiate into appropriate regenerative lineages (i.e. neurons, astrocytes, and oligodendrocytes). The requirements listed above may be addressed, in part, by selecting an appropriate carrier for the cell therapy as described herein.

The design requirements for an acceptable cell carrier are to: 1) prevent cell settling during transportation and injection storage, 2) promote cell survival, 3) facilitate administration of homogeneous therapeutic cell suspensions, and 4) enable rapid clearance by the body following injection so as not to interfere with cellular integration with surrounding tissue. Importantly, combining the cells with the carrier should not adversely affect cell viability during mixing, or upon injection or at the transplantation site.

Sedimentation of therapeutic cells due to cellular aggregation occurs in storage solutions for stem cells, stem cell delivery systems and stem cell delivery compositions. The present invention enables the preparation of a storage stable injectable medium of suspended stem cells for the manufacture, storage and delivery of stem cells to a target delivery site in the spinal cord of a human or animal in various diagnostic and therapeutic settings.

The present invention describes injectable mediums comprising, for example, human neural stem cells suspended in a liquid medium that offers numerous advantageous properties by preventing cellular aggregation and minimizing the disruption and sedimentation of stem cells during transport, storage and administration of such injectable medium. The injectable mediums comprising human neural stem cells are suitable for the delivery of the human neural stem cells to the CNS, particularly to the spinal cord, of a human or animal subject, in various diagnostic and therapeutic settings. The present invention is also suitable in applications such as cell therapy and tissue engineering (such as 3-D printed cellular constructs).

While the delivery and administration of human neural stem cells is a preferred embodiment of the present invention, other types of therapeutic cells may be administered using the methods and compositions described herein. Therapeutic cells may also include neural stem cells, pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem cells, the differentiated progeny of any of the above, genetically modified cells, or other cell types.

The described injectable mediums may also be utilized to deliver therapeutic substances, alone, or more preferably together with therapeutic cells to the CNS, especially to the spinal cord. Various neurotropic factors are contemplated in the art. Therapeutic agents that may be incorporated into the liquid composition comprising hyaluronic acid include: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other like therapeutic agents.

The administration of trophic and growth factors such as erythropoietin (EPO), brain-derived neurotrophic factor (BDNF), nerve growth factor (NGF), fibroblast growth factor (FGF) and epidermal growth factor (EGF) appear to play an important role in in-vitro and in-vivo survival and differentiation of stem cells (Erickson et al., Roles of insulin and transferrin in neural progenitor survival and proliferation. J. Neurosci. Res. 2008 Feb. 21; Bossolasco et al., Neuro-glial differentiation of human bone marrow stem cells in vitro., Exp. Neural., 2005 June; 193(2):312-25). Nerve growth factor (NGF) appears to influence grafted tissue in the CNS. Mahoney. et al. (1999). Med. Sci. 96:4536-4539.

Other regulatory agents comprising various growth factors including insulin-like growth factor-I (IGF-I) and basic fibroblast growth factor (bFGF) also regulate the survival and differentiation of nerve cells during the development of the peripheral and central nervous systems. IGF-I promotes differentiation of post-mitotic mammalian CNS neuronal stem cells. Arsenijevic, et al. (1998) J. Neurosci. 18:2118-2128. Similarly, neurotrophins have been shown to be important for nerve growth during development. Tucker, et al. (2001), Nature Neurosci., 4:29-37). GAP-43 and CAP-23 act to promote regeneration of injured axons and may support regeneration in the spinal cord and CNS. Bomze et al. (2001) Nature Neurosci. 4:38-43 and Woolf et al. (2001) Nature Neurosci. 4:7-9. Cocktails of growth factors may be used to further increase cell survival, neuronal differentiation, axon extension, and synapse formation (Lu, et al., Long-Distance Growth and Connectivity of Neural Stem Cells after Severe Spinal Cord Injury. Cell, 2012, Sep. 14; 150(6): 1264-1273).

Given the various problems of delivering a trail of neural stem cells into the spinal cord parenchyma by administration and delivery systems known in the art, there is still a need for an injectable medium comprising human neural stem cells suspended in a liquid medium that offers numerous advantageous properties by preventing cellular aggregation and minimizing the disruption and sedimentation of stem cells during transport, storage and administration of such liquid medium.

The settling of therapeutic cells and/or therapeutic substances or diagnostic substances or other injectable medium may also impose a problem when transporting syriges pre-loaded with cells. In therapeutic delivery applications, including cell delivery or the creation of therapeutic trails of cells, cells may settle in the delivery syringe prior to or during injection. This may result in problems such as inaccurate dosing, inhomogeneous cell delivery, and potentially cell death during injection. This problem is exacerbated in cell delivery applications where the duration of the operation is comparable with the settling time of the of therapeutic cells and/or therapeutic substances or diagnostic substances in the injectable medium.

The shipping of containers, such as a syringe, pre-loaded with the cell suspension may be difficult due to cell settling during the shipping process. To circumvent this problem, additional handling steps may be required to prepare the cells prior to administration at the time of surgery. Preventing or reducing settling or aggregation of the therapeutic elements, such as cells, will therefore address or reduce the effect that the above-discussed problems. Reducing settling may include prolonging the characteristics settling time for the therapeutic cells and/or therapeutic substances or diagnostic substances or other injectable medium.

Unique advantages provided by the present invention include improved properties of the cellular suspensions after a prolonged storage period, improved cellular homogeneity during and after injection, improved clearance from the central nervous system in a comparably short amount of time after injection, and potentially the facilitation of the suspended stem cells to interact via receptors on the neural stem cell surface to promote cell survival after storage and\or injection of the compositions of the invention.

In a first aspect the present invention provides an injectable medium comprising therapeutic cells, and optionally therapeutic or diagnostic substances, suitable for injection into an anatomical space of a human or animal subject, comprising:
(a) therapeutic cells, and optionally therapeutic or diagnostic substances;
(b) a pharmaceutically acceptable diluent comprising hyaluronic acid;
wherein the injectable medium has a storage modulus within the range of 5-25 Pa.

In a second aspect the present invention provides an injectable medium comprising therapeutic cells, and optionally therapeutic or diagnostic substances, suitable for injection into an anatomical space of a human or animal subject, wherein
(a) therapeutic cells, and optionally therapeutic or diagnostic substances;
(b) a pharmaceutically acceptable diluent comprising hyaluronic acid;
wherein the hyaluronic acid is formulated at a concentration of about 0.5 wt. % to 1 wt. % in the injectable medium; and further wherein the hyaluronic acid has a molecular weight of about 700 kDa to about 1,900 kDa; and wherein the injectable medium has a storage modulus within the range of 5-25 Pa.

In a third aspect, the present invention provides an injectable medium comprising neural stem cells, and optionally therapeutic or diagnostic substances, suitable for injection into an anatomical space of a human or animal subject, comprising:
(a) human neural stem cells, and optionally therapeutic or diagnostic substances;
(b) a pharmaceutically acceptable diluent comprising hyaluronic acid;
wherein the injectable medium has a storage modulus within the range of 5-25 Pa.

In a fourth aspect, the present invention provides an injectable medium comprising neural stem cells, and optionally therapeutic or diagnostic substances, suitable for injection into an anatomical space of a human or animal subject, comprising:
(a) human neural stem cells, and optionally therapeutic or diagnostic substances;
(b) a pharmaceutically acceptable diluent comprising hyaluronic acid;
wherein the hyaluronic acid is formulated at a concentration of about 0.5 wt. % to 1 wt. % in the injectable medium; and further wherein the hyaluronic acid has a molecular weight of about 700 kDa to about 1,900 kDa; and wherein the injectable medium has a storage modulus within the range of 5-25 Pa.

In a fifth aspect, the present invention provides a method of preparing an injectable medium comprising therapeutic cells, and optionally one or more therapeutic or diagnostic substance, suitable for injection into an anatomical space of a human or animal subject, comprising the steps of:
(a) introducing into a sterilized vial a desired quantity of therapeutic cells, and optionally one or more therapeutic or diagnostic substance;
(b) adding to the vial a pharmaceutically acceptable diluent comprising hyaluronic acid;
(c) mixing the above composition until a substantially uniform suspension is obtained having a storage modulus within the range of 5-25 Pa.

In a sixth aspect, the present invention provides a method of preparing an injectable medium comprising therapeutic cells, and optionally one or more therapeutic or diagnostic substance, suitable for injection into an anatomical space of a human or animal subject, comprising the steps of:
(a) introducing into a sterilized vial a desired quantity of therapeutic cells, and optionally one or more therapeutic or diagnostic substance;
(b) adding to the vial a pharmaceutically acceptable diluent comprising hyaluronic acid;
(c) resuspending the therapeutic cells in the diluent by agitating the vial;
wherein the hyaluronic acid is formulated at a concentration of about 0.5 wt. % to 1 wt. % in the injectable medium; and further wherein the hyaluronic acid has a molecular weight of about 700 kDa to about 1,900 kDa;
(d) mixing the above composition until a substantially uniform suspension is obtained having a storage modulus within the range of 5-25 Pa.

In a seventh aspect, the present invention provides a method of preparing an injectable medium comprising neural stem cells, and optionally therapeutic or diagnostic substances suitable for injection into an anatomical space of a human or animal subject, comprising the steps of:
(a) introducing into a sterilized vial a desired quantity of human neural stem cells;
(b) adding to the vial a pharmaceutically acceptable diluent comprising hyaluronic acid;
(c) mixing the above composition until a substantially uniform suspension is obtained having a storage modulus within the range of 5-25 Pa.

In an eighth aspect, the present invention provides a method of preparing an injectable medium comprising neural stem cells, and optionally therapeutic or diagnostic substances suitable for injection into an anatomical space of a human or animal subject, comprising the steps of:
(a) introducing into a sterilized vial a desired quantity of human neural stem cells;
(b) adding to the vial a pharmaceutically acceptable diluent comprising hyaluronic acid;
(c) resuspending the human neural stem cells in the diluent by agitating the vial;
wherein the hyaluronic acid is formulated at a concentration of about 0.5 wt. % to 1 wt. % in the injectable medium; and further wherein the hyaluronic acid has a molecular weight of about 700 kDa to about 1,900 kDa;
(d) mixing the above composition until a substantially uniform suspension is obtained having a storage modulus within the range of 5-25 Pa.

In a ninth aspect of the present invention, the therapeutic cells of the first, second, fifth and sixth aspects include pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem, genetically modified cells, neural precursor cells of the forebrain, midbrain, hindbrain, spinal cord, neural crest, and retinal precursors isolated from developing tissue, and the undifferentiated and differentiated progeny of any of the above.

In a tenth aspect of the present invention, the human neural stem cells of the third, fourth, seventh and eight aspects may be undifferentiated or differentiated cells.

In an eleventh aspect of the present invention, the cells of the first to tenth aspects may be delivered as spheres, aggregates or single cell suspensions.

In a twelfth aspect of the present invention, the pharmaceutically acceptable diluent of the first to eleventh aspect may be divalent ion-free buffed salt solution; phosphate buffered saline; cell culture medium, isotonic saline, hanks buffered salt solution, HEPES buffered salt solution, and artificial cerebrospinal fluid.

In a thirteenth aspect of the present invention, the pharmaceutically acceptable diluent of the first to twelfth aspects may further comprise ascorbic acid, glucose, or glutamine.

In a fourteenth aspect of the present invention, the injectable medium of the first to thirteenth aspects may further comprise a neuroprotective, angiogenic, anti-angiogenic or neuroregenerative pharmaceutical substance.

In an fifteenth aspect of the present invention, the injectable medium of the first to fourteenth aspects may further comprise at least one factor capable of stimulating endogenous stem cells.

In a sixteenth aspect of the present invention, the injectable medium of the first to fifteenth aspects may further comprise a drug and/or growth factor selected from the group consisting of: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

In a seventeenth aspect of the present invention, the injectable medium of any of the preceding aspects may further comprise a regulatory agent, as described herein.

In an eighteenth aspect of the present invention, the injectable medium of any of the preceding aspects may further comprise a therapeutic substance, as described herein.

In a nineteenth aspect of the present invention, the injectable medium of any of the preceding aspects may be injected into the spinal cord of a subject using a suitable injection system that deposits one or more trails of therapeutic cells, including human neural stem cells within the spinal cord of the subject.

In a twentieth aspect of the present invention, the injectable medium of any of the preceding aspects may be injected into the brain of a subject using a suitable injection system that deposits one or more trails of therapeutic cells, including human neural stem cells within the brain of the subject

In yet another aspect of the present invention, the method of injecting one or more trails of neural stem cells within the spinal cord of the subject may be used to treat a spinal cord injury, condition or disease.

In still yet another aspect of the present invention, the injectable medium comprises human neural stem cells suspended in a carrier comprising high molecular weight hyaluronic acid at a concentration of about 0.5 weight % to about 1 weight % in the injectable medium; wherein the hyaluronic acid has a molecular weight of about 700 kDa to about 1,900 kDa; and wherein the composition enables the human neural stem cells to be suspended uniformly for up to two days, up to three days, up to four days or up to five days.

In a further aspect of the present invention, a kit suitable for injecting a trail of neural stem cells into the spinal cord of a subject using a suitable injection system is provided, wherein the kit comprises hyaluronic acid at a concentration of 0.5 weight % to 1 weight % in an injectable medium wherein the hyaluronic acid has a molecular weight of about 700 kDa to about 1,900 kDa; and wherein the composition enables the human neural stem cells to be suspended uniformly for up to two days, up to three days, up to four days or up to five days.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other objects, features and advantages will be apparent from the following more particular description of the embodiments described in detail below, as illustrated in the accompanying drawings, in which like reference characters refer to the same parts throughout the different views. The following detailed description, given by way of example, but not intended to limit the present invention to the specific embodiments described, may best be understood in conjunction with the accompanying drawings. The drawings are not necessarily to scale, emphasis instead being placed upon illustrating the principles of the embodiments.

FIG. 1 is a temperature sweep of 0.75 wt. % hyaluronic acid (HA) in phosphate buffered saline (PBS).

FIG. 2 is a rheology plot of HA solutions with varying molecular weight (MW) (1.0 wt. % in PBS).

FIG. 3 is a rheology plot of HA solutions with varying weight percentage in PBS (1.01-1.8 MDa MW HA).

FIGS. 4 A&B depicts a rat neural stem cell (NSC) suspension in HA of varying molecular weight. (A) T=0; (B) T=72 h.

FIGS. 5 A&B depicts a rat NSC suspension in high MW HA (1.01-1.8 MDa) of varying weight percentage in PBS. (A) T=0; (B) T=72 h.

FIGS. 6 A&B depicts (A) a trypan blue human NSC viability following mixing with PBS or HA (1.01-1.8 MDa MW; 0.75 wt. %-1.0 wt. % or 2 wt. % in PBS) and (B) flow through a fine gauge needle (60 cm long 29G nitinol needle attached to a 30 cm long 30G polychlorotrifluoroethene tube).

FIGS. 7 A&B depicts an injection of NSC trails into collagen spinal cord mimics. Rat NSCs (100,000 cells/uL) were suspended in (A) PBS or (B) 0.75 wt. % HA (1.01-1.8 MDa).

FIG. 8 depicts clearance of high molecular weight HA (˜1.5 MDa) from rat brain over six days. HA was labeled with biotinylated HA binding protein (HABP) and Alexa Flour-555 streptavidin. The asterisk (*) denotes areas of HABP signal.

FIG. 9 depicts HA-FITC (˜1.5 MDa) binding to NSCs. Human NSCs were incubated with 5 mg/mL HA-FITC in PBS for 30 min @ 37° C.

FIGS. 10A&B. (A) depicts human NSC suspension in high MW HA (1.01-1.8 MDa) or PBS post mixing, after 24 hours, after 48 hours, and after 5 days at 4° C. (B) depicts trypan blue cell viability of human NSCs following storage in high MW HA (1.01-1.8 MDa) or PBS for 0 hours, 24 hours, 48 hours, and 5 days, following injection through a 29G needle.

FIGS. 11A&B. (A) depicts trypan blue cell viability of human NSCs following storage in high MW HA (1.01-1.8 MDa) or PBS for 24 h and injection through a 29G needle. (B) depicts tryptan blue cell viability of human NSCs following storage in high MW HA (1.01-1.8 MDa) or PBS for 48 h and injection through a 29 g needle. The cell suspensions were partially ejected from the syringe following 24 hours in storageand then ejected again after 48 hours in storage.

FIG. 12 shows images of rat NSCs suspended in various mediums for up to one hour according to one embodiment.

FIG. 13 depicts the delivery of rat NSCs into a spinal cord mimetic gel according to an embodiment.

FIG. 14 depicts, according to an embodiment, microtubule associated protein-2 (MAP2) staining of human NSCs in a HA carrier injected into a spinal cord mimetic gel in vitro and cultured for 7 days.

FIG. 15 shows a trail of rat NSCs in a HA carrier delivered into a rat spinal cord according to an embodiment.

FIG. 16 shows images of rat NSCs in a HA carrier stored in a syringe for up to 40 h according to an embodiment.

FIG. 17 shows trypan blue staining of rat NSCs stored in a HA carrier at 4° C. according to one embodiment.

FIGS. 18A&B are photographs of two views of trails of a high MW HA and methylene blue mixture injected at an angle in a “tent” formation around a prophetic injection site.

FIGS. 19A-C are graphical representations of the injection angles to be used in Example 1.

FIG. 20 depicts a therapeutic trail delivery system according to some embodiments of the present invention assembled on an optional mobile cart.

FIG. 21 is a graphical representation of the surgical procedure set-up of experimental trail injection device 900 as it will be employed in Example 1.

FIG. 22 depicts an injection dispensing device apparatus comprising a syringe containing cells and/or other therapeutic substances, a guide needle, an injection needle and an adjustable goniometer for pitch adjustment as well as a motorized injection needle assembly terminating in an injection needle.

FIGS. 23A&B are is an illustrations of a programmable controller of an embodiment of the present invention. A=right view; B=left view.

FIG. 24 is a graphic representation of the angle measurements in accordance with the injection of a 20 mm trail of the liquid composition of HA and methylene blue in accordance with Example 3.

FIG. 25 depicts the testing setup for injection device 900 used in this Example 3.

FIGS. 26A-C are images of methylene blue trails from a liquid composition comprising HA and methylene blue injected into an agarose slab at a setting of 4 mm at an injection angle of 11.5°, 6 mm at an injection angle of 17.5° and at 8 mm at an injection angle of 23.6° in accordance with Example 3.

FIG. 27 is an image of a guide needle positioned at the surface of an agarose gel slab and an injection needle penetrating the agarose gel slab at a setting of 8 mm and an injection angle of 23.6° yielding a trail of 8 mm in accordance with Example 3.

FIG. 28 depicts a rheology plot showing the effect of human NSC addition on the strain-dependent mechanical properties (storage & loss modulus) of the composition.

FIG. 29 depicts depicts a rheology plot showing the effect of human NSC addition on the temperature-dependent mechanical properties (storage & loss modulus) of the composition.

FIG. 30 STEM121 (green) and doublecortin (DCX; red) immunohistochemical staining of human NSCs delivered in a HA carrier into a nude rat spinal cord after one month. Lower case letters in the three bottom panels of FIG. 30 are enlargements of the respective indicated areas of the top panel.

FIG. 31 depicts human NSCs delivered in a HA carrier into a nude rat spinal cord after three months. The cells were labeled for makers of human cytoplasm (STEM121), human astrocytes (STEM123), and axons (TUJ1).

FIG. 32 depicts survival of a trial of human NSCs (STEM121 positive) delivered in a HA carrier through a spinal cord contusion injury in a nude rat after three months.

FIG. 33 depicts cross-sections and longitudinal sections of a trail of human NSCs (STEM121 positive) delivered in a HA carrier into a porcine spinal cord after one week.

FIGS. 34A&B show (B) photographs of syringes pre-filled with the NSC-HA composition and (A) stored in one embodiment of a pre-filled syringe carrier.

### DETAILED DESCRIPTION

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. Instead, the proper scope of the embodiments is defined by the appended claims. Further, stating that a feature may exist indicates that the feature may exist in one or more embodiments.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or. Moreover, if these terms are used, a subset of a set may include one or more than one, including all members of the set.

The present invention provides methods and pharmaceutical compositions to improve cell-based therapies used to regenerate tissues, including neural tissue, that has been damaged or is undergoing degeneration by any degenerative, for example, neurodegenerative disorder. In particular, the present invention provides methods and pharmaceutical compositions to improve cell-based therapies used to regenerate neural tissue in the central nervous system, in particular the spinal cord of a human or a mammal.

As used herein, “central nervous system” (CNS) refers to the brain and spinal cord and associated tissues.

As used herein, “effective amount” of cells and/or agent is an amount sufficient to prevent, treat, reduce and/or ameliorate the symptoms, neural damage and/or underlying causes of any of the neurodegenerative disorders of the CNS described herein. An effective amount may include by way of example and not limitation, about 10,000 to about 100 million therapeutic cells per subject.

An efficacious dosage range for the delivery of regulatory agents may be 0.01 mg/ml to 1 g/ml of injectable medium.

An efficacious dosage range of therapeutic cells may be 10,000 to 1×10⁹ cells per subject.

The term “gene therapy” is used throughout the specification to describe the transfer of new genetic information into cells for the therapeutic treatment of diseases or disorders. The gene products produced by the genetically modified cells can be harvested in vitro or the cells can be used as vehicles for in vivo delivery of the gene products (i.e., gene therapy). The gene therapy may also be delivered to the subject's endogenous cells.

The terms “grafting” and “transplanting” and “graft” and “transplantation” are used throughout the specification synonymously to describe the process by which cells of the subject invention are delivered to the anatomical site where the cells are intended to exhibit a favorable effect, such as repairing damage to a patient's central nervous system (which can reduce a cognitive or behavioral deficit caused by the damage), treating a neurodegenerative disease or treating the effects of nerve damage caused by stroke, cardiovascular disease, a heart attack or physical injury or trauma or genetic damage or environmental insult to the brain and/or spinal cord, caused by, for example, an accident or other activity.

An “injectable medium” in the context of the present invention is a type of heterogeneous mixture of cells in a liquid or gelatinous medium. A “homogenous” mixture occurs when the cells are “substantially uniformly distributed in the injectable medium”. The terms “homogenous” and “substantially uniformly distributed” are used interchangeably.

The term “neurodegenerative disease” is used herein to describe a disease which is caused by damage to the central nervous system and which damage can be reduced and/or alleviated through transplantation of neural cells according to the present invention to damaged areas of the brain and/or spinal cord of the patient. Exemplary neurodegenerative diseases which may be treated using the neural cells and methods according to the present invention include for example, Parkinson's disease, Huntington's disease, amyotrophic lateral sclerosis (Lou Gehrig's disease), Alzheimer's disease, Rett Syndrome, spinal muscular atrophy, lysosomal storage disease (“white matter disease” or glial/demyelination disease, as described, for example by Folkerth, J. Neuropath. Exp. Neuro., September 1999, 58:9), including Sanfilippo, Gaucher disease, Tay Sachs disease (beta hexosaminidase deficiency), other genetic diseases, multiple sclerosis, brain injury or trauma caused by ischemia, accidents, environmental insult, etc., spinal cord damage, ataxia and alcoholism. In addition, the present invention may be used to reduce and/or eliminate the effects on the central nervous system of a stroke or a heart attack in a subject, which is otherwise caused by lack of blood flow or ischemia to a site in the brain of the subject or which has occurred from physical injury to the brain and/or spinal cord. Neurodegenerative diseases also include neurodevelopmental disorders including for example, autism and related neurological diseases such as schizophrenia, among numerous others.

As used herein, the term “patient” or “subject” refers to a mammal including a non-primate (e.g. a cow, pig, horse, dog, cat, rat and mouse) and a primate (e.g. a monkey and human), and preferably a human.

As used herein, “regulatory agent” refers to any molecule having a growth, proliferative, differentiative, or trophic effect on a transplanted donor cell of the present invention or subject's endogenous cells. Regulatory agents may include, without limitation, include: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), and calpain inhibitors. Moreover, regulatory agents may include without limitation pharmaceutical substances such as anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other like therapeutic agents.

As used herein, “therapeutic cells(s)” comprises at least one cell or type of cell, for example, and not by way of limitation, a neural stem cell to be administered to the CNS, in particular to the spinal cord, of a human or mammalian subject undergoing cell-replacement or gene therapy. Therapeutic cell(s) may be derived from any source and may be at various stages of developmental differentiation as long as the therapeutic cell(s) are sufficient to prevent or reduce the morphological and/or behavioral neurological symptoms of the neurodegenerative disorder or injury being treated with cell-replacement therapy according to the present invention. Therapeutic cell(s) may be either heterologous (allogeneic or xenogeneic) or autologous to the host. By heterologous it is intended that the therapeutic cell is derived from a mammal other than the subject, while an autologous therapeutic cell is derived from the subject, optionally manipulated ex vivo, and transported back into an anatomical site of the subject, for example the CNS, more particularly the spinal cord of a patient, by methods of the present invention.

Examples of therapeutic substances/agents include vectors; living cells; therapeutic cells (stem cells, progenitor cells, cells having a capability to differentiate into a specific type of cell, predifferentiated cells, or cells emitting or triggering emission of a healing biochemical after delivery into a subject; viruses having therapeutic potential; drugs; pharmaceutical agents; one or more pharmacologically active ingredients disposed in a delivery vehicle, a shell, or a casing; drug carrier systems; capsules containing drugs; nanocrystals or other nano- or microparticles for example theranostic nanoparticles, gold nanoparticles, silver nanoparticles, iron oxide nanoparticles, other inorganic or organic nanoparticles, biochemicals susceptible to various delivery conditions or forms of delivery stress; functionalized molecules; proteins; carbohydrate compounds and materials; small molecules compounds that may be sensitive to some aspect of delivery; and masses of cells that have begun to form into a tissue structure; nanostructures or other constructs (synthetic, biological, or some combination thereof) carrying pharmaceutical or cellular payloads, etc., without being exhaustive.

As used herein, “therapeutic substances comprise “regulatory agents,” “growth factors.” “therapeutic agents” and other pharmaceutical substances known in the art.

Various terms, including “treat,” “therapy” and “therapeutic,” as used herein, refer to alleviate, slow the progression, prophylaxis, attenuation or cure of a damaged or degenerating CNS involving loss or death of CNS cells, in particular for treatment of SCI.

### Pharmaceutical Compositions.

The present invention relates to methods of formulating an injectable medium comprising neural stem cells and hyaluronic acid in a pharmaceutically acceptable diluent for administration via a suitable injection system into the spinal cord of a subject.

Methods for formulating an injectable medium are generally known in the art. See the following reference for a thorough discussion of formulation and selection of pharmaceutically acceptable carriers, stabilizers, and other substances recognized in the art as a “pharmaceutically acceptable excipient.” Remington's Pharmaceutical Sciences (18.sup.th ed.; Mack Publishing Company, Eaton, Pa., 1990), herein incorporated by reference. The worker skilled in the art can choose excipients recognized in the art as GRAS (generally regarded as safe) for use as a pharmaceutically acceptable excipient in a topical or parenteral formulations.

An important consideration when selecting an injectable medium against those compositions known in the art is the risk of the composition posing toxicity issues or acting as a barrier to cellular integration once injected into certain organs. While it may be possible to prepare and inject an injectable medium comprising hyaluronic acid, neural stem cells and other therapeutic agents, it is preferred to avoid incorporating chemicals into said composition such as methylcellulose that exhibit minimal short-term adverse effects but have unknown long-term biological consequences in central nervous system applications (LaPlaca et al., Biomaterials, 2001).

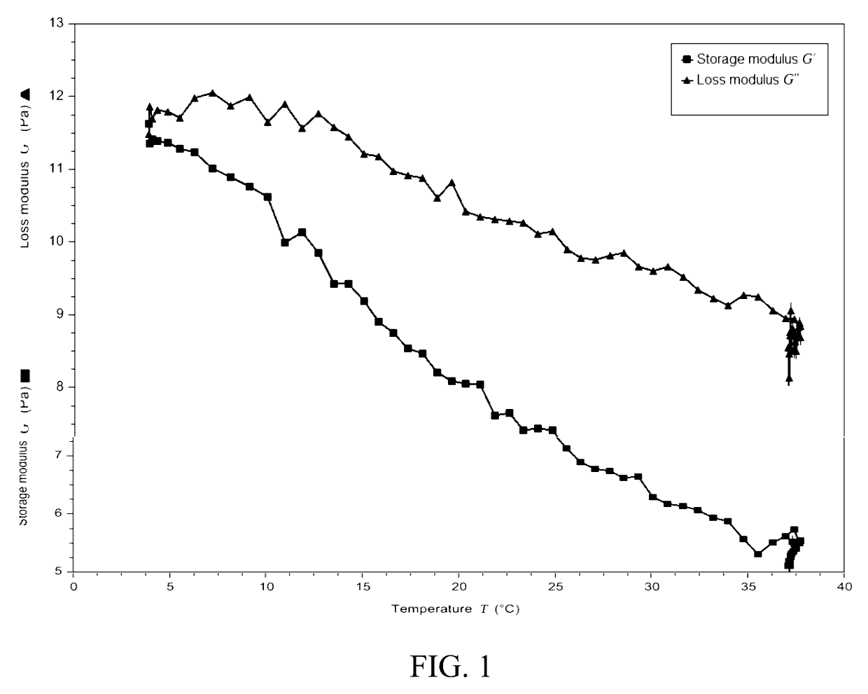

Another consideration when selecting an injectable medium formulation in view of those compositions known in the art, is the ability of the injectable medium to maintain an injectable or preferred storage modulus when the composition is exposed to room temperature after being stored on ice or a cool temperature of the like for transportation. U.S. Pat. No. 7,767,656, discloses compositions that become more viscous when the temperature of the solution is warmed. Problems arise when gels of high viscosities become difficult to inject through a fine gauge needle required to deliver such compositions to an animal. This problem is exacerbated when flowing the gel through the extended lengths of tubing used in clinical cell injection apparatuses. High viscosity gels may induce excessive shear forces on the cells and result in cell death. In the present invention, referring to FIG. 1, a composition comprising a high molecular weight hyaluronic acid, specifically greater than 700 kDa to 1,900 kDa, in a final solution concentration of 0.75 weight % to 1.0 weight % in a buffered salt solution, can form a composition which becomes less viscous when warmed to room temperature and therefore more injectable.

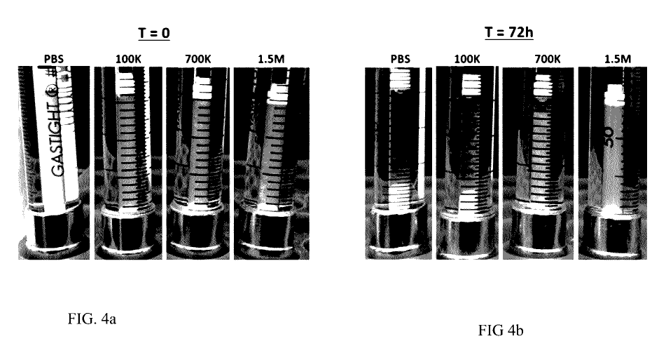

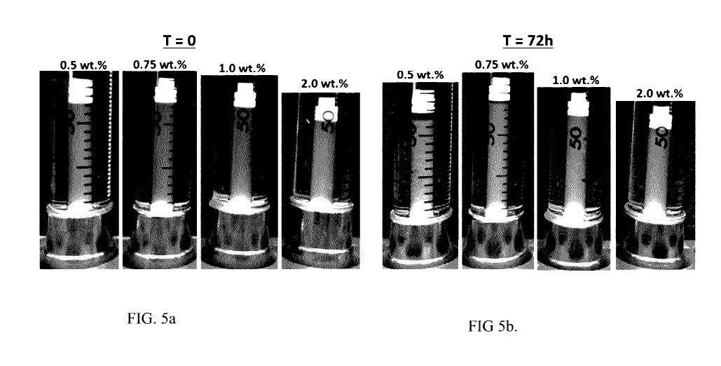

Another consideration when selecting an injectable medium is the ability of the injectable medium to maintain a homogeneous cellular suspension for purposes of transportation, storage and use. An injectable medium comprising a high molecular weight hyaluronic acid, specifically those greater than about 700 kDa to about 1,900 kDa, in a final solution concentration of about 0.75 weight % to about 1.0 weight % in a buffered salt solution, can form an injectable medium which consistently retains homogeneous cell suspension for up to two day, up to three days, up to four days or up to five days, as compared to those that include a lesser molecular weight hyaluronic acid (FIG. 4) and/or a lesser final solution concentration than about 0.75 weight % to about 1.0 weight % of hyaluronic acid in a buffered salt solution (FIG. 5).

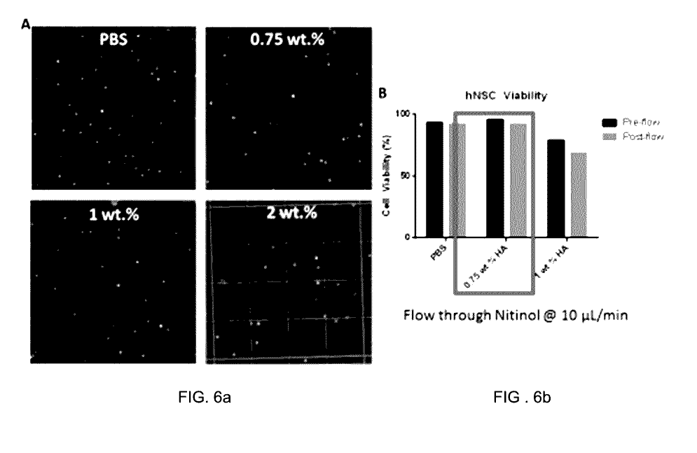

Compositions with lesser weight % or lower molecular weights are insufficient to maintain a homogenous suspension. Importantly, the composition should maintain cell viability during the mixing process and after subsequent injection through a fine gauge needle. Compositions with 1.01-1.8 MDa molecular weight, but formulated at a higher weight %, result in excessive cell death upon mixing (FIG. 6). In other words, in order to ensure that the composition maintains cell viability during mixing, homogeneous cellular suspension until injection, and cell viability after injection, the composition should utilize a high molecular weight hyaluronic acid, specifically those greater than about 700 kDa, in a final solution concentration of about 0.75 weight % to about 1.0 weight % in a buffered salt solution.

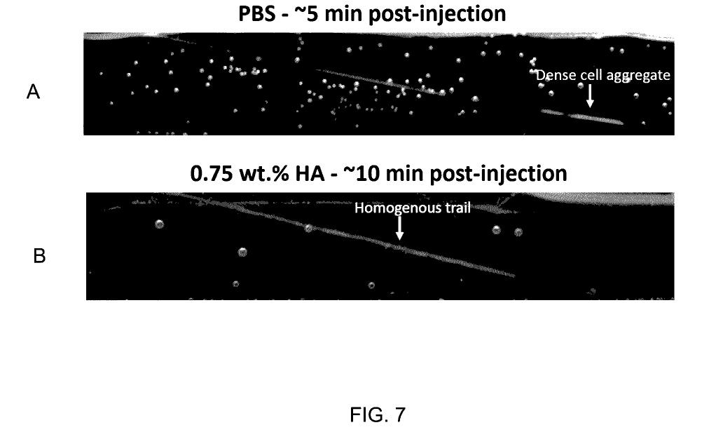

The disclosed injectable medium also enables the delivery of homogenous cell deposits. These deposits, such as “trails” of cells, may be useful for applications such as spinal cord regeneration. Homogenous trails of neural stem cell-derived neurons may form relays that reconnect severed spinal circuits. Referring to FIG. 7, a composition comprising a high molecular weight hyaluronic acid in a buffed salt solution and neural stem cells was injected into a collagen spinal cord mimic and the injection of this composition resulted in a uniform trail of cells. In contrast, a suspension of cells in a buffered salt solution alone resulted in a cellular aggregate\pellet near the bottom of the injection tract.

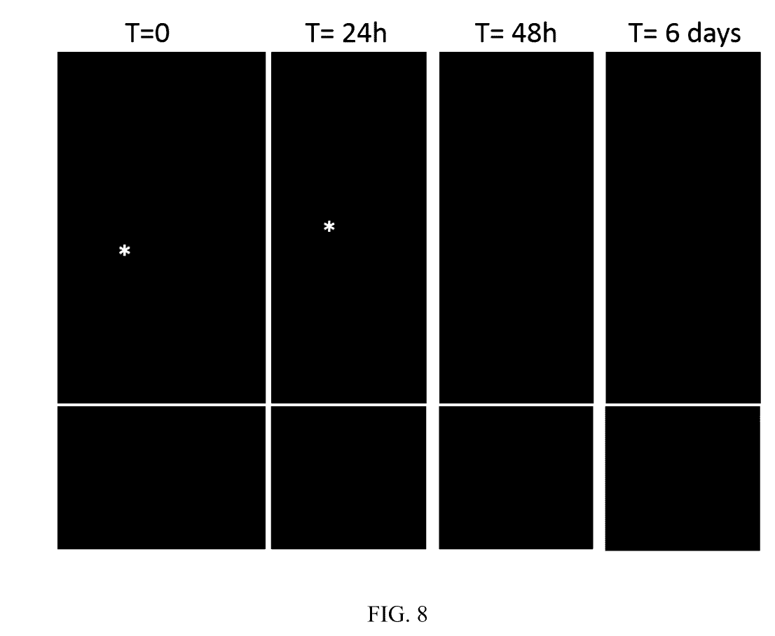

Compositions known in the art, such as those that utilize methylcellulose, do not degrade from the central nervous system (“CNS”) tissue. Blends of hyaluronic acid and methylcellulose are not fully degraded even after 30 days in vitro (Gupta, Tator, & Shoichet., Biomaterials, 2006). In vivo compositions of hyaluronic acid and methylcellulose persist in the intrathecal space for at least 4 days (Kang et al., Tissue Engineering: Part A, 2009). In addition, the clearance profile in the intrathecal space may be accelerated due to cerebrospinal fluid flow compared to within CNS tissue itself. Compositions which do not degrade remain at the site of injection and may prohibit other cellular mechanisms, such as integration into surrounding tissue, from taking place. Referring to FIG. 8, a composition comprising a high molecular weight hyaluronic acid, specifically those greater than about 700 kDa in a final solution concentration of about 0.75 weight % to about 1.0 weight % in a buffered salt solution can form a composition which degrades from the CNS tissue in roughly two days, experimentally determined by immunohistochemically labeling the high-molecular weight hyaluronic acid.

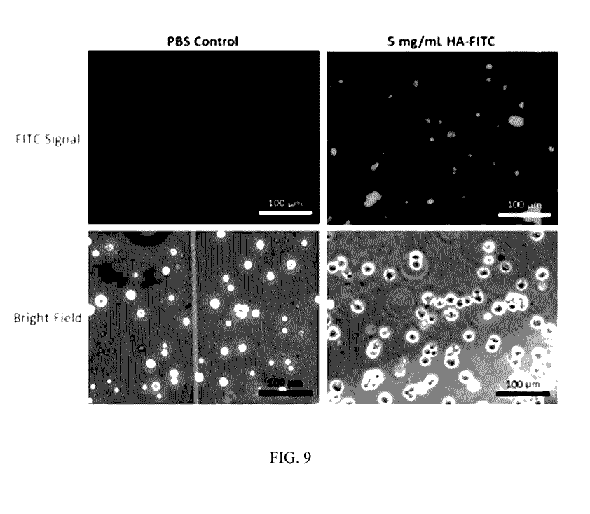

Compositions known in the art that include polyethylene glycol (PEG) or cellulose (Lampe et al., J Biomed Mater Res A, 2010), are also not known to interact with receptors on the surface of the neural stem cells to promote cell survival after injection. Referring to FIG. 9, a composition comprising a high molecular weight hyaluronic acid, specifically those greater than about 700 kDa, in a final solution concentration of about 0.75 weight % to about 1.0 weight % in a buffered salt solution is shown to form a composition wherein hyaluronic acid binds to the surface of the neural stem cells. Activation of CD44 may be beneficial for the survival of the stem cells.

The injectable medium may also contain conventional pharmaceutical additives, such as preservatives, stabilizing agents, wetting, or emulsifying agents, salts for adjusting osmotic pressure, buffers, and the like. The carrier may be hypotonic or isotonic with body fluids and have a pH within the range of 4.5-8.5.

Other acceptable components in the injectable medium comprise, without limitation, isotonicity-modifying agents such as water, saline, and buffers including phosphate, citrate, succinate, acetic acid, and other organic acids or their salts. The use of buffers, stabilizers, reducing agents, anti-oxidants and chelating agents in the preparation of protein-based compositions, particularly pharmaceutical compositions, is well known in the art. See, Wang et al. (1980) J. Parent. Drug Assn, 34(6):452-462; Wang et al. (1988) J. Parent. Sci. Tech. 42:S4-S26 (Supplement); Lachman et al. (1968) Drug and Cosmetic Industry 102(1):36-38, 40, and 146-148; Akers (1988) J. Parent. Sci. Tech. 36(5):222-228; and Methods in Enzymology, Vol. XXV, ed. Colowick and Kaplan, “Reduction of Disulfide Bonds in Proteins with Dithiothreitol,” by Konigsberg, pp. 185-188.

Various embodiments of the injectable medium of the present invention comprise suitable buffers such as acetate, adipate, benzoate, citrate, lactate, maleate, phosphate, tartrate, borate, tri(hydroxymethyl aminomethane), succinate, glycine, histidine, the salts of various amino acids, or the like, or combinations thereof. See Wang (1980) supra at page 455. Suitable salts and isotonicifiers include sodium chloride, dextrose, mannitol, sucrose, trehalose, or the like.

Various embodiments employ a settling reduction technique by creating a suspension of the therapeutic elements (e.g., cells) in a viscous liquid or shear-thinning polymer such as hyaluronic acid. The vicious liquid or shear thinning polymer may be formulated in a divalent ion-free buffer solution such as phosphate buffered saline. In some embodiments the weight percentage of hyaluronic acid in the divalent ion-free carrier may be from about 0.5 wt. % to about 1 wt. %. The average molecular weight of the hyaluronic acid may be larger than about 1000 kDa. The skilled worker will understand that the weight percentage of hyaluronic acid and the molecular weight thereof may be adjusted to achieve an injectable medium having a storage modulus within the range of 5-25 Pa. This technique may also be used to transport therapeutic cells and/or therapeutic or diagnostic substances pre-loaded into a syringe to maintain homogeneity of the cells during shipping.

A “pharmaceutically acceptable diluent” is intended to comprise a carrier that is conventionally used in the art to facilitate the storage, administration, and/or the biological activity of therapeutic cells, regulatory agents, within an injectable medium of the present invention. A pharmaceutical acceptable diluent may thus also comprise therapeutic cells, regulatory agents, and pharmaceutically acceptable excipients.

A suitable carrier should be stable, i.e., incapable of reacting with other ingredients in the formulation. It should not produce significant local or systemic adverse effect in recipients at the dosages and concentrations employed for treatment. Suitable carriers are generally known in the art and can be found in Remington's Pharmaceutical Sciences (18.sup.th ed.; Mack Publishing Company, Eaton, Pa., 1990), herein incorporated by reference.

Divalent ion-free buffed salt solution; phosphate buffered saline; cell culture medium, isotonic saline, hanks buffered salt solution, HEPES buffered salt solution, and artificial cerebrospinal fluid, water, saline, aqueous dextrose, and glycols are preferred liquid carriers, particularly (when isotonic) for solutions.

Other forms of compositions for administration of therapeutic cells and/or pharmaceutical compositions or elements thereof include a suspension of a particulate, such as an emulsion, a liposome, or in a sustained-release form to prolong the presence of the pharmaceutically active agent in a subject.

The compositions can be prepared under sterile conditions and can be sterilized by means known in the art.

The present invention also relates to methods comprising the step of administering neural stem cells in a suspension comprising hyaluronic acid and a pharmaceutically acceptable diluent for the injection of trails of neural stem cells into the spinal cord of a subject.

The invention further relates to compositions comprising an injectable medium of neural stem cells, hyaluronic acid and a pharmaceutically acceptable diluent, wherein the injectable medium is a uniform suspension having a storage modulus in the range of 5-25 Pa.

The invention further relates to compositions comprising an injectable medium of neural stem cells, hyaluronic acid and a pharmaceutically acceptable diluent, wherein the hyaluronic acid is formulated at a concentration of about 0.5 wt. % to about 1 wt. % in the injectable medium; and further wherein the hyaluronic acid has a molecular weight of about 700 kDa to about 1,900 kDa; and wherein the injectable medium is uniform and has a storage modulus in the range of 5-25 Pa.

Cell therapy compositions may be administered at ambient temperatures; however, such compositions are frequently stored at hypothermic (2° to 8° C.) conditions for early phase clinical trials, either of allogeneic or xenogeneic therapies. It is also more likely to be used for autologous cell therapies, which utilize patients' own cells as a starting material. Cryopreservation of NSC cell populations is likely to be necessary for long-term storage of cell therapies prior to administration.

The mechanical properties of different viscoelastic materials can be compared by measuring the storage and loss moduli. The storage modulus describes the elastic property of viscoelastic materials and the loss modulus describes the viscous property. Comparison of the storage moduli or loss moduli is performed at a strain within the linear viscoelastic region of the material, a region where the storage and loss moduli are independent of strain. Furthermore, storage and loss moduli of many viscoelastic materials are dependent on temperature, so comparisons between compositions needs to be made within the same temperature range. The storage moduli described herein are measured on a TA Ares G2 rheometer fitted with a 25 mm parallel plate fixture (0.5 mm gap). The testing conditions for the claimed storage moduli (i.e. 5-25 Pa) are a temperature of 20° C. and a strain of 1% at a frequency of 1 Hz. This strain (1%) is within the linear viscoelastic region of the compositions.

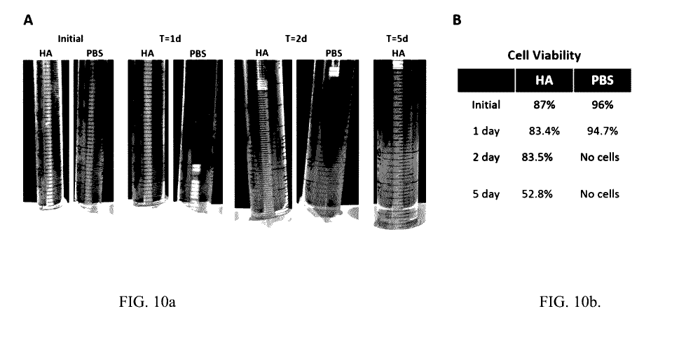

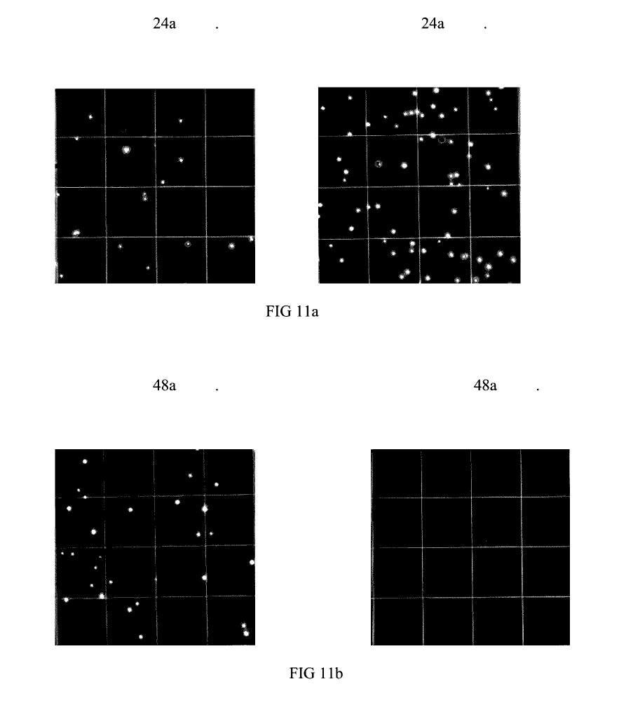

Shelf-life studies have assessed the viability of the therapeutic and commercially manufactured neural stem cell line (StemPro Neural Stem Cells, ThermoFisher Scientific), formulated in PBS. In general, the cells will are stored at 2° to 8° C. prior to administration. However, the cells may be at ambient temperature during administration, and this temperature shift has been taken into account during the shelf-life assays. The data from all experiments employing 24-96 hours storage at 2° to 8° C. followed by a temperature shift to ambient are presented in FIG. 10, which clearly demonstrates excellent viable shelf-life afforded by HA in PBS over saline formulations for two days, and >50% viability even after 5 days of storage. Importantly, the HA composition maintained a homogenous cell suspension that could be sampled over the 5 day storage period. In contrast, as shown in FIG. 10b and FIG. 11, when the cells were stored in PBS they rapidly pelleted and were all extruded from the syringe following the first sampling at 24 h. This cell settling resulted in low cell dosing after 24 h of storage.

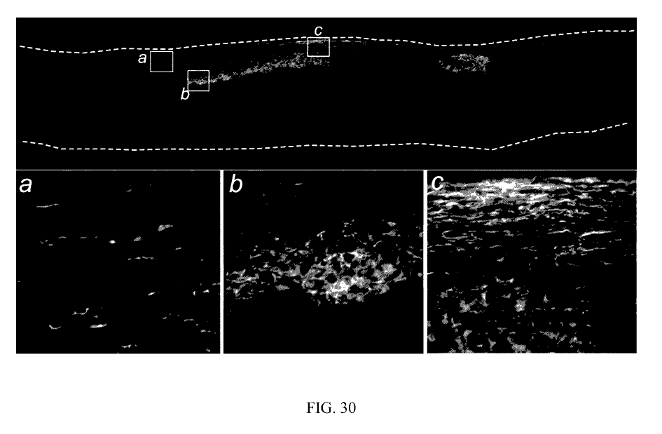

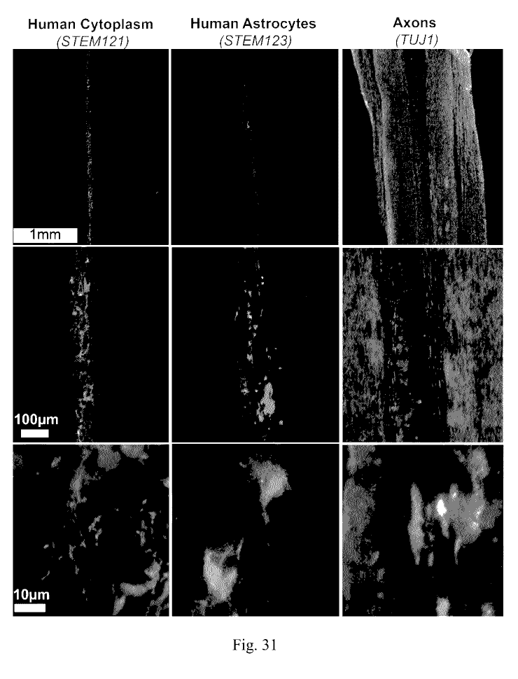

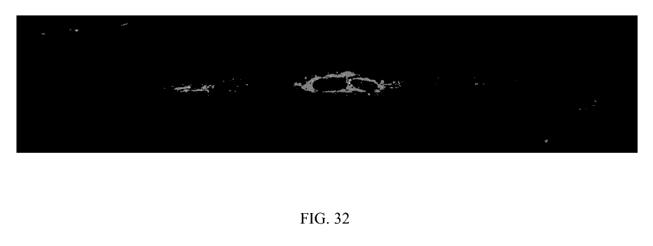

Beyond practical cell viability and stability concerns, a cell carrier needs to facilitate cell survival, integration, and differentiation (in the case of stem cells). HA is a natural and biocompatible biomaterial with a ˜48 hour clearance time (FIG. 8). These properties may facilitate in vivo cell survival, integration, and differentiation. Compositions of neural stem cells in the hyaluronic acid carrier have been delivered into the spinal cord of nude rats (uninjured and injured) and pigs. FIGS. 30, 31, 32, and 33 show in vivo long term survival and differentiation of the human neural stem cells. Importantly, the neural stem cells delivered in the hyaluronic acid carrier survive even in the difficult environment of a contused spinal cord (FIG. 32). Articles and Methods of Manufacture

The present invention also includes an article of manufacture providing therapeutic cells and/or injectable medium comprising therapeutic cells and/or components of the injectable medium of the present invention to the CNS, in particular, administration to the spinal cord of a human or mammal.

The article of manufacture may include a vial or other container that contains an injectable medium suitable for the present method together with any carrier, either dried or in liquid form.

The instructions contain information such as sufficient dosage and administration information so as to allow the subject or a worker in the field to administer the therapeutic cells and/or therapeutic or diagnostic substance comprising of the present invention.

In particular, the article of manufacture may comprise a pre-filled syringe containing therapeutic cells, and optionally therapeutic or diagnostic substances; or a prefilled syringe containing therapeutic cells, and optionally therapeutic or diagnostic substances, wherein the syringe is utilized in the injection system described in Provisional Patent Application No. 62/384,505 filed Sep. 7, 2016, and non-provisional application filed contemporaneously herewith entitled METHODS AND SYSTEMS FOR DELIVERY OF A TRAIL OF A THERAPEUTIC SUBSTANCE INTO AN ANATOMICAL SPACE which disclosure is incorporated herein by reference in its entirety.

### Neural Stem Cells

As used herein, the term “neural stem cells” has its usual meaning in the art and refers to cells which have the capacity to differentiate into cells with different neural phenotypes. The term also includes cells referred to as “progenitor” cells.” A “neuronal progenitor cell” is an undifferentiated cell that is derived from a neural stem cell and which has committed to a particular path of differentiation, and under appropriate conditions will differentiate into neurons or glial cells (astrocytes, oligodendrocytes).

The use of such multipotent neuronal cell lineages for transplantation is known in the art. See, for example, Snyder et al. (1992) Cell 68:33, where multipotent neuronal cell lines have been grafted into the rat cerebellum to form neurons and glial cells. See also, Campbell et al. (1995) Neuron 15:1259-1273; Fishell et al. (1995) Development 121:803-812; and, Olsson et al. (1995) Eur. J. Neurosci. 10:71-85; Donnelly et al. (2012) Stem Cell Research & Therapy, 3:24; and Martinez-Morales et al. (2013) Stem Cell Rev and Rep 9:685.

The neural stem cells of the invention are by definition multipotent, i.e. they are capable of differentiating into a number of neural cell types (e.g. neurons, glial cells and oligodendrocytes). A neural stem cell is defined herein as a multipotent cell that is an immature and uncommitted multipotent cell that exists in the nervous system (Ourednik et al. (1999) Clinical Genetics 56:267-278). Neural stem cells may differentiate under suitable conditions to neurons, glial cells, and oligodendrocytes. See also the following with regard to the understanding of neural stem cells in the art. McInnes et al. (1999) Clin. Genet. 56:267-278.

The term “pluripotent” is well recognized in the art and refers to the ability of the cells to develop into more than two types of cells. Pluripotent neuroepithelial cells are capable of differentiating into neural cells of a different phenotype. The term “progenitor” is well recognized in the art and refers to the ability of the cells to differentiate into a defined phenotype. Neural progenitor cells can only differentiate into cells with a neural phenotype.

“NSCs” as used in the present specification, describes a cell that is capable of undergoing greater than 20-30 cell divisions while maintaining the potency to generate both neurons and glia. Preferably, the cells are capable of undergoing greater than 40, more preferably greater than 50, and most preferably unlimited cell divisions. Neural stem cells are capable of dividing either symmetrically, or asymmetrically. In the former instance, the neural stem cell divides to form two daughter neural stem cells or two committed progenitors. When dividing asymmetrically, the neural stem cell divides to form one daughter neural stem cell, and one committed progenitor (e.g. either a neuron or a glial progenitor).

Neural stem cells of the invention can be derived from, inter alia, humans, primates, rodents, and birds. Preferably, the neural stem cells are derived from humans. Neural stem cells can be derived directly from embryos, from adult tissue, from fetal tissue, or from embryonic stem (ES) cells (either wild-type or genetically modified ES cells). Preferably, the neural stem cells of the invention are derived from human fetal cells. The cells may also be derived originally from embryonic stem cells, which have been induced to undergo differentiation into neural stem cells. The cells may also be derived from induced pluripotent cells, i.e. genetically reprogrammed cells which, through the use of transcription factors, have been induced into a pluripotent phenotype. The NSCs may also be derived by direct transdifferentiation.

Neural stem cells (NSCs) can be derived from the ventricular and hippocampal regions of the fetal and adult brain and can be isolated by sorting brain cells using specific cell surface markers (Uchida et al., 2000) and expanded in defined mediums with additional growth factors (Reynolds and Weiss, 1992; Carpenter et al., 1999; Vescovi et al., 1999). NSCs can also be generated from pluripotent cells directly, such as embryonic stem cells (Conti et al. 2005). Although such cells are suitable, the capacity for expansion of NSCs is limited to a relatively small number of cell passages, necessitating additional technologies to permit a scalable, standardized, stable and, therefore, commercial application. The cells may therefore be modified to permit multiple cell passages or may be cultured under conditions which promote multiple cell passaging. Suitable conditions will be apparent to the skilled person.

It is possible to derive neural stem cells of the invention from a wide variety of sources. For example, neural stem cells can be derived directly from embryos, from adult tissue, from foetal tissue, or from embryonic stem (ES) cells (either wild-type or genetically modified ES cells). Preferably, the neural stem cells of the invention are derived from mouse or human ES cells, or are derived from mouse or human foetal cells. Neural stem cells of the invention can be derived from, inter alia, humans, primates, rodents, and birds. Preferably, the neural stem cells are derived from mammals, especially mice, rats and humans. Neural stem cells may be obtained, for example, by the procedures of U.S. Pat. No. 9,309,495, which is incorporated herein by reference in its entirety.

Neural stem cells may be derived from human foetal brain and maintained in tissue culture as described by Pollock et al (2006). Human mesenchymal stem cells isolated from bone marrow withdrawn from the posterior iliac crest of the pelvic bone of normal volunteers, were obtained from Lonza (Catalogue number: PT-2501) and cultured as recommended by the manufacture using their proprietary medium MSCGM (Mesenchymal Growth Medium).

Human neural stem cells (StemPro, ThermoFisher Scientific) were passaged as semi-adherent cultures on culture dishes (CellBIND, Corning Inc.) in a humidified 37° C. incubator. The cell culture medium consisted of KnockOut™ DMEM\F-12 (ThermoFisher Scientific) supplemented with StemPro Neural Supplement (ThermoFisher Scientific), basic fibroblast growth factor (ThermoFisher Scientific), epidermal growth factor (ThermoFisher Scientific), fetal bovine serum (HyClone), heparin sodium salt (Sigma Aldirch), and Pen Strep (Life Technologies). Cell culture medium was replaced every 3-4 days. Cells were dissociated with Accutase (StemPro, ThermoFisher Scientific) and passaged as necessary.

Rat hippocampal Green Fluorescent Protein Neural Stem Cells (Millipore) were passaged as adherent cultures on culture dishes (CellBIND, Corning Inc.) in a humidified 37° C. incubator. The cell culture medium consisted of KnockOut™ DM EM\F-12 (ThermoFisher Scientific) supplemented with StemPro Neural Supplement (ThermoFisher Scientific), basic fibroblast growth factor (ThermoFisher Scientific), Leukemia Inhibitory Factor (Life Technologies), heparin sodium salt (Sigma Aldirch), Puromycin (Sigma Aldrich), and Pen Strep (Life Technologies). Cell culture medium was replaced every 3-4 days. Cells were dissociated with Accutase (StemPro, ThermoFisher Scientific) and passaged as necessary.

### Hyaluronic Acid

As used herein, “hyaluronic acid (HA)” refers to hyaluronan, hyaluronate, subunits of HA that have been tested and found suitable for use in a mammal, including a human.

Hyaluronic acid is the major component of the extracellular matrix (ECM) found in large quantities in skin. It is also the major physiological component of the articular cartilage matrix and is particularly abundant in synovial fluid. Hyaluronic acid, in its acid or salt form, is a biomaterial largely used as injectable filler material for augmentation of dermal tissue or other soft tissue like gingival tissue, filler material for ophthalmic applications, as a viscosupplement osteoarthritis treatment.

Chemically, hyaluronic acid is a linear non-sulfated glycosaminoglycan composed of repeating units of D-glucuronic acid and N-acetyl-D-glucosamine (Tammi R., Agren U M., Tuhkanen A L., Tammi M. Hyaluronan metabolism in skin. Progress in Histochemistry & Cytochemistry 29 (2): 1.-81, 1994).

HA is highly viscous, highly electronegative and highly hydrophilic. Various methods for the isolation, purification and fractionation of HA are known to those skilled in the art. In addition, molecular mass fractions of purified HA can be purchased from commercial sources including, but not limited to, Fluka Chemical Corporation (Ronkonkoma, N.Y., USA), Genzyme Corporation (Cambridge, Mass., USA), Lifecore Inc. (Chaska, Minn., USA), Hyal Pharmaceutical Corporation (Mississauga, Ontario, Canada) and Bioniche Life Sciences, Inc. (Belleville, Ontario, Canada).

The present invention will now be described with reference to the Figures and Examples.

In FIG. 1 is shown a temperature-sweep rheology plot of a 0.75 wt. % HA solution in PBS (Kikkoman Biochemifa, 1200-1900 kDa MW). Testing was performed on a TA Ares G2 rheometer fitted with a 25 mm parallel plate fixture (0.5 mm gap). Testing conditions: Temperature ramp from 4° C. to 37° C. (1% strain, 1 Hz). The temperature ramp shows a reduction in storage modulus with increasing temperature. The storage modulus is ˜11.5 Pa at 4° C. and ˜5 Pa at 37° C. Reduced storage modulus at injection temperature (20-25° C.) may improve the ease of injection, reduce cell shear, and improve cell viability during injection.

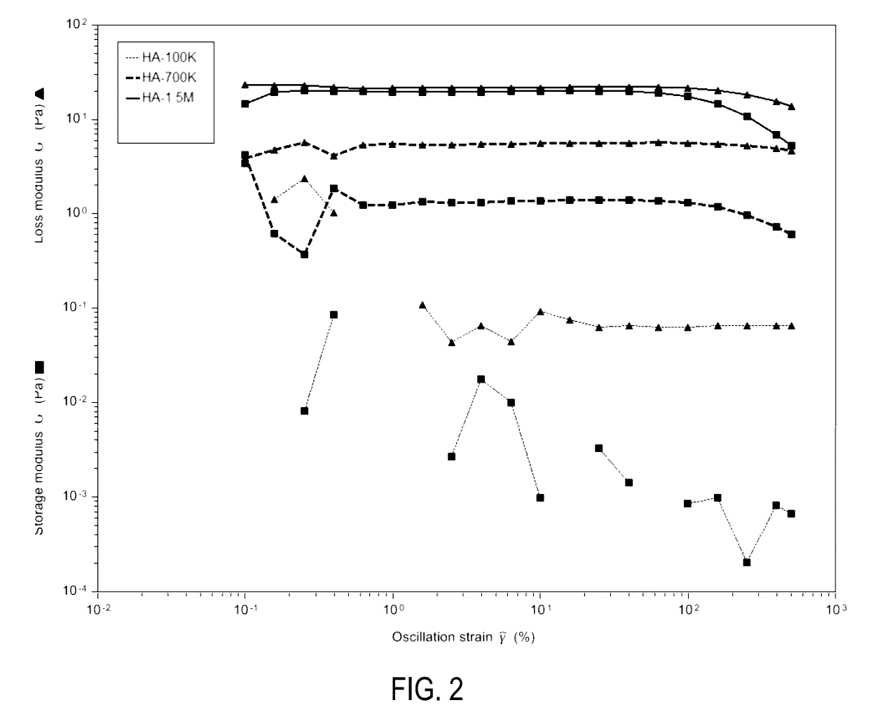

In FIG. 2 is shown a rheology plot of HA solutions with varying molecular weights (MW) (1.0 wt. % in PBS). Testing was performed on a TA Ares G2 rheometer fitted with a 25 mm parallel plate fixture (0.5 mm gap). Testing conditions included Strain sweep @ 20° C. from 0.1 to 500% strain, 1 Hz. Samples: HA-100K (100-150 kDa MW HA, LifeCore Biomedical), HA-700K (500-749 kDa MW HA, LifeCore Biomedical), HA-1.5M (1.01-1.8 MDa MW HA, LifeCore Biomedical). The strain sweep shows increasing storage modulus with increasing MW of HA. This is likely a result of the increased entanglements with high MW HA. This strain sweep testing indicated that a 100 kDa MW for HA is insufficient to induce molecular entanglements and provide mixture viscosity necessary for cell suspension.

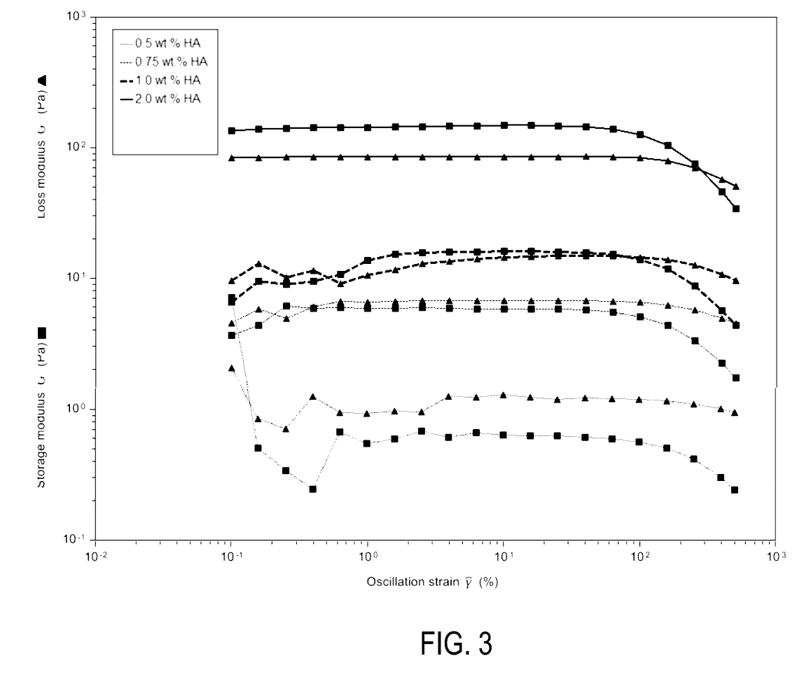

Referring to FIG. 3 is shown a rheology plot of HA solutions with varying weight percentage (1.01-1.8 MDa MW HA). Testing was performed on a TA Ares G2 rheometer fitted with a 25 mm parallel plate fixture (0.5 mm gap). Testing conditions: Strain sweep @ 20° C. from 0.1 to 500% strain, 1 Hz. Samples: HA-1.5M (1.01-1.8 MDa MW HA, LifeCore Biomedical) in PBS. The storage modulus of the HA is lower than or equal to the loss modulus for weight percentages of 1 wt. % or below. The storage modulus is greater than the loss modulus for 2 wt. % HA. This indicates that 1 wt. % and below compositions are more liquid-like (prior to the addition of cells) and the 2 wt. % HA is more gel or solid-like. These physical properties are important for cell viability during mixing and injection, as cells may experience less shear-induced cell death in a liquid-like formulation with a lower storage modulus.

FIG. 4 depicts a neural stem cell (NSC) suspension in HA of varying MW. All syringes contain 100,000 rat neural stem cells per microliter in either: PBS (Phosphate buffered saline), HA-100K (100-150 kDa MW HA, LifeCore Biomedical), HA-700K (500-749 kDa MW HA, LifeCore Biomedical), or HA-1.5M (1.01-1.8 MDa MW HA, LifeCore Biomedical). Increasing the weight percentage of HA in the PBS solution likely results in increased molecular entanglement and thus increased storage modulus. Furthermore, 1 wt. % and 2 wt. % HA solutions exhibited storage moduli that were higher than the respective loss moduli, indicating that 1 wt. % and 2 wt. % solutions behave more as gels than viscous liquids. The strain sweep testing of varying weight percentage HA solutions was used to determine storage modulus ranges sufficient for homogenous cell suspension. Surprisingly, as seen in the subsequent figures, a gel-like mixture (storage>loss modulus) is unnecessary to achieve long term homogenous cell suspension. This is advantageous both because it is easier to produce, mix, and handle liquid solutions than solid gels and injecting less viscous solutions may prevent cell death due to shear. Crosslinked HA gels are typically used in the art, and these crosslinked HA gels exhibit deficiencies in the aforementioned areas.

FIG. 5 depicts a neural stem cell (NSC) suspension in high MW HA (1.01-1.8 MDa) of varying weight percentage in PBS. All syringes contain 100,000 rat neural stem cells per microliter in either: PBS (or HA-1.5M (1.01-1.8 MDa MW HA, LifeCore Biomedical) of increasing weight percentage (0.5 to 2.0 wt. %). All solutions are homogenous initially (T=0). However by 72 h (and within a few minutes for PBS), cell aggregation and settling was visible in the PBS and low molecular weight HA (100K) syringe. The 700K MW HA somewhat prevents cell settling however a clear band of settled cells was visible under the plunger of the syringe. The syringe high molecular weight HA (1.5M) was completely uniform after 72 h of storage.

FIG. 6 depicts a trypan blue human NSC viability following mixing with PBS or HA (1.01-1.8 MDa MW; 0.75-2.0 wt. %) and flow through a fine gauge needle (60 cm long 29G nitinol attached to a 30 cm long 30G polychlorotrifluoroethene tube. Flow rate=10 uL/min). (A) Representative images of trypan blue stained NSCs following mixing with HA. (B) Quantification of NSC viability following flow through a 29G nitinol needle. Mixing human NSCs in PBS or 0.75 wt. % resulted in little loss of cell viability (both ˜93-95%; pre-flow). Mixing human NSCs in 1.0 wt. % HA resulted in a reduction of cell viability (78.9% viable) and large loss of cell viability in the 2.0 wt. % HA (<50% viable), likely due to the increased viscosity of the 1.0 wt. % and 2.0 wt. % suspension (gel-like suspensions; storage modulus>loss modulus). To test viability after injection, the injectable medium comprising cells was flowed through fine tubing. Minimal loss of cell viability was observed in the PBS and 0.75 wt. % HA suspension (1.2% and 3.5% reduction, respectively), however additional loss of cell viability was observed in the 1.0 wt. % HA group (10.4% loss). This shows that the 0.75 wt. % HA formulation is ideal to prevent cell death during mixing and injection while providing for a stable cell suspension. Viscous gel-like suspensions (storage modulus>loss modulus) as observed for 1.0 wt. % and 2.0 wt. % HA resulted in substantial cell death upon cell suspension preparation and subsequent use (injection).

FIG. 7 depicts an injection of rat NSC trails into collagen spinal cord mimics. Rat neural stem cells (100,000 cells/μL) were suspended in PBS or 0.75 wt. % HA (HA-1.5M). The suspension was injected into 0.5 wt. % collagen gels using a nitinol needle-based injection apparatus that extruded 40 mm long trails of cells. Images of the resulting cell trails were recorded 10 minutes post injection %). Substantial cell pelleting and aggregation was observed in the cell trail injection in PBS, with many cells pelleting towards the bottom of the angled trail. In contrast, a uniform trail of cells was observed when the cells were injected in 0.75 wt. % HA. This shows the utility of high molecular weight 0.75 wt. % HA compositions for injecting homogenous trails of cells.

FIG. 8 depicts clearance of FITC-labeled high molecular weight HA (˜1.5 MDa) from rat brain. FITC images (green) detect the signal from the FITC label covalently conjugated to the HA. Due to residual diffuse free-dye in retained in brain tissue (not shown), the brain sections were subsequently labeled with biotinylated HA Binding Protein (HABP; Amsbio) and Streptavidin-Alexa Fluor 555 (red; ThermoFisher Scientific). As exemplified in the HABP stained sections, the HA signal greatly decreased from time 0 to 24 h (asterisk depicts HA signal). Minimal signal was detected at 48 hours post injection. A 48 hour resorption rate of the HA carrier may facilitate cellular integration at the treatment site.

FIG. 9 depicts HA-FITC (˜1.5 MDa) binding to NSCs. Human NSCs were incubated with 5 mg/mL HA-FITC in PBS for 30 min @ 37° C. The cells were then washed three times with PBS and imaged on a hemocytometer slide. Control cells were incubated in PBS without HA-FITC. Green (HA-FITC) signal was detected in a majority of the human NSCs. HA attachment or uptake is likely due to interactions of CD44 receptors on the cell surface with HA. This receptor-mediated interaction may facilitate cell survival during storage and after administration.

FIG. 10 depicts 12 million human neural stem cells suspended in 120 μL HA (0.75 wt. %) or PBS and loaded into 100 μL Hamilton syringes (n=2 for the HA group), as depicted in FIG. 10b. The syringes were stored upright at 4 C. After 24 h, the syringes were imaged, as depicted in FIG. 10a, then syringes were connected to 29G nitinol needle (1 inch length). The needle was primed with cells at 20 μL/min until cell suspension was visible extruding from the needle. 10 μL of cells was ejected at a rate of 10 μL/min into 1 mL of PBS. The solution was gently mixed, combined 1:1 with Trypan Blue and imaged on a hemocytometer. After taking the 10 μL sample, the syringes were re-capped and placed back into storage at 4 C. After an additional 24 h (48 h total), the cell sampling procedure with 29G needle was repeated. Cell aggregation and settling was visible in the PBS suspension at 24 h while the HA suspension was homogenous for up to 5 days. Furthermore, the cell viability in the HA suspension was maintained for up to 5 days, albeit with ˜50% viability at the 5 day time point. The pelleting of the cells in PBS resulted in all of the cells being ejected during the first sampling procedure (t=24 h, FIG. 11a). No cells remained in the PBS syringe for sampling at the 48 h time point (FIG. 11b). The uniform suspension in the HA syringes allowed for reproducible sampling of the cells. This highlights how inadequate cell suspension results in inconsistent dosing after storage, whereby an initial injection releases the highly concentrated cell pellet and subsequent doses have little or no cells.

FIG. 11 depicts cell viability data with reference to the procedures in FIG. 10. At 48 h there were no cells detected in the PBS syringe, as depicted in FIG. 11b as compared to FIG. 11a. This may be due to cell settling, where all the settled cells were ejected during the priming\sampling procedure at the 24 h time point. In contrast, the cells suspended in HA delivered homogenous cell samples both at 24 h (FIG. 11a) and at 48 h (FIG. 11b). As described in FIG. 10, this highlights how inadequate cell suspension results in inconsistent dosing after storage, whereby an initial injection releases the highly concentrated cell pellet and subsequent doses have little or no cells. Stable cell suspension in the HA composition allows for consistent cell dosing after storage.

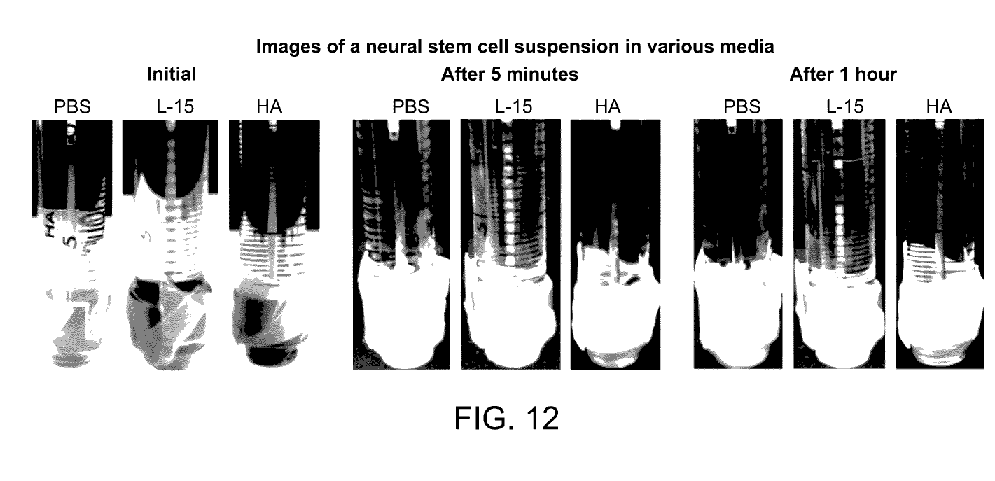

FIG. 12 depicts rat neural stem cell suspensions (100,000 cells\μL) in PBS, Leibovitz L-15 medium (L-15), or in a HA suspension (0.5 wt. % HA in L-15 medium, Kikkoman Biochemifa HA 1200-1900 kDa MW, in this embodiment). The cells may settle in phosphate buffered saline (PBS) after 5 minutes and may aggregate within 5 minutes. However, the cells are uniformly suspended in HA for up to one hour. Some embodiments utilize this property for the delivery of a homogenous cell suspension when the delivery time is up to one hour or potentially longer. This figure also shows that divalent ion free diluents may prevent cell aggregation. The cells settled to the bottom of the syringe in PBS (divalent ion free) but formed large aggregates in L-15 medium (contains divalent ions). The addition of HA to L-15 medium prevents cell aggregation in this embodiment. Formulating the HA in PBS instead of L-15 may further reduce cell aggregation.

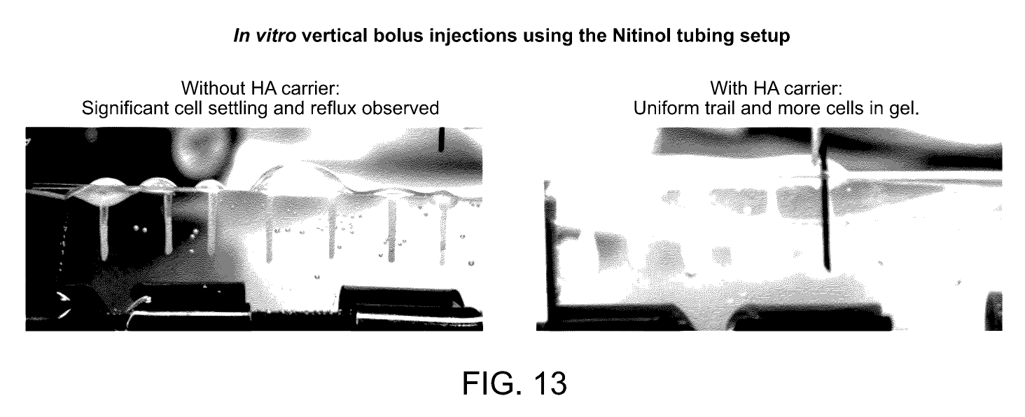

FIG. 13 depicts the delivery of neural stem cells into a spinal cord mimetic gel according to an embodiment. When the cells are delivered without a HA carrier (in L-15 medium), cells may aggregate to the bottom of the needle track. When cells are injected in an HA carrier (0.75 wt. % Kikkoman Biochemifa HA, 1200-1900 kDa MW in a divalent ion-free PBS), however, the cells are uniformly distributed along the injection track. Furthermore, more of the cells are deposited within the gel when injected in the HA carrier. Taken together, this data shows that the HA carrier increases the homogeneity of cell deposition along a needle tract and may reduce cellular reflux, thus resulting in more accurate cell dosing.

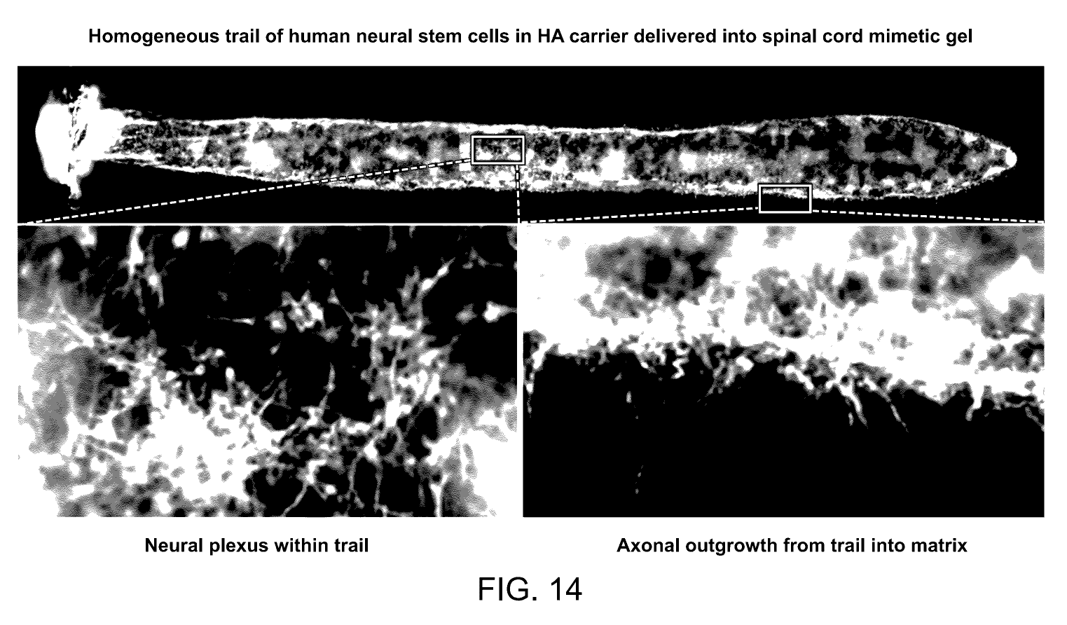

FIG. 14 depicts, according to an embodiment, microtubule associated protein-2 (MAP2) staining of human neural stem cells in a HA carrier (0.75 wt. % Kikkoman Biochemifa HA, 1200-1900 kDa MVV) injected into a spinal cord mimetic gel in vitro. After one week in culture, the cells express survive and pre-neuronal markers in the uniform cell trail. Outgrowth of neuronal projections is also visible at the boarders of the trail. In some embodiments, this situation indicates that the viscosity of the hyaluronic acid formulation (0.75 wt. %) permits the survival and outgrowth of human neural stem cells in vitro. Therefore the HA composition is a suitable carrier for delivering neural stem cells in CNS applications.

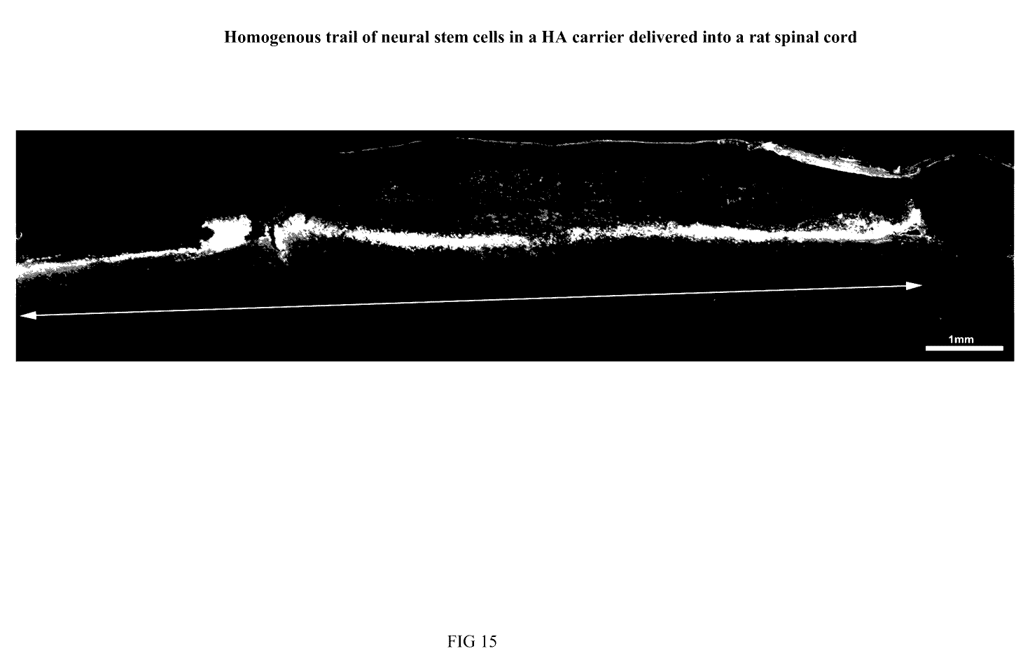

FIG. 15 shows a trail of GFP-expressing rat neural stem cells in a HA carrier (0.75 wt. % HA, (Kikkoman Biochemifa, 1200-1900 kDa MW) delivered into a rat spinal cord according to an embodiment. In this embodiment, the HA formulation facilitated a homogenous cell trail in vivo and the viscosity of the HA solution permitted cell survival (4 days post injection). Therefore, the HA composition is a suitable delivery vehicle for uniform cell trails in vivo.

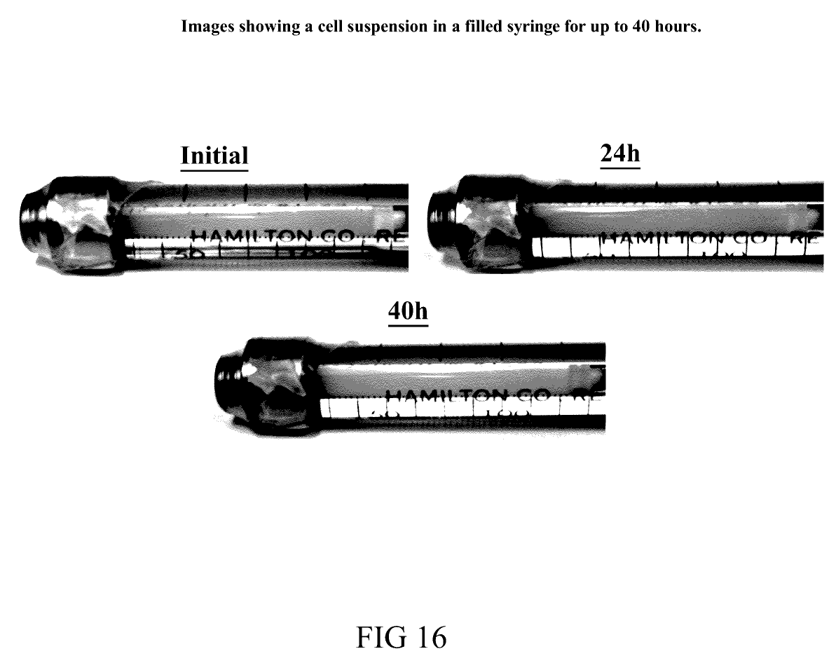

FIG. 16 shows images of rat neural stem cells in HA (0.75 wt. % in PBS) (Kikkoman Biochemifa, 1200-1900 kDa MVV) stored in a syringe for up to 40 h according to an embodiment. This time course may be representative of the time necessary to ship a pre-filled syringe of cells to the site of application (for example, hospital). The images in FIG. 16 show that a homogenous cell suspension may be maintained in hyaluronic acid for up to 40 h and demonstrate the utility of this formulation as a cell carrier for shipping applications.

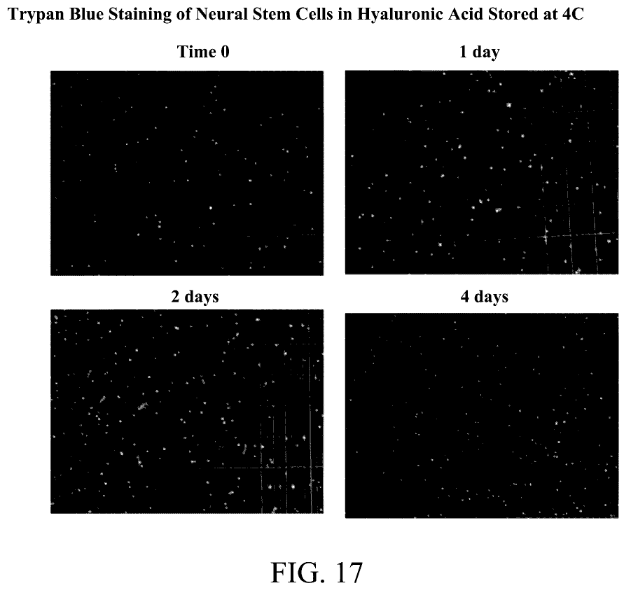

FIG. 17 shows trypan blue staining of rat neural stem cells stored in a 0.75 wt. % HA carrier (Kikkoman Biochemifa, 1200-1900 kDa MW) at 4° C. according to one embodiment. This time course of images may demonstrate that the cells, shown as bright spots, may remain viable over the course of 4 days when stored in the HA. This result may indicate that the HA weight percentage and molecular weight used, correlating to a solution storage modulus of ˜10 Pa, may maintain the viability of cells and may be suitable for the maintaining the viability of cells. The homogenous cell suspension shown in FIG. 16 coupled with the cell viability shown in FIG. 17 may demonstrate the utility of the viscous liquid or shear-thinning polymer for cell transportation.

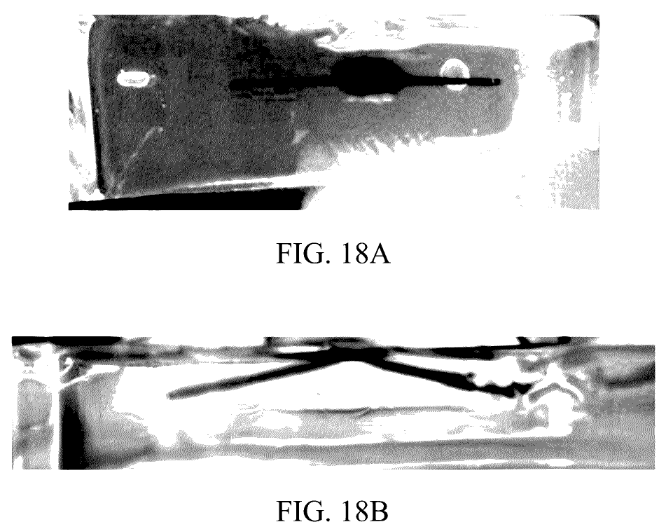

FIGS. 18a and 18b illustrate trails injected at an angle in a “tent” formation around a prophetic injection site. Two opposing 2-cm long trails injected at 10 degree angles into a 0.6 wt. % agarose gel slab. The trails are composed of 0.75 wt. % HA in PBS and methylene blue was added for visualization purposes. FIG. 18a is a top view and 18b is a side view illustrating the angular injections and the described “tent” feature which may be used to inject a trail of cells and/or therapeutic substances proximal to an injury site in the spinal cord. With regard to the injection procedure, reference may be made to Example 1 above. FIGS. 18a and 18b are photographs of methylene blue stained hyaluronic acid trails injected at an angle in a “tent” formation around a prophetic injection site. Furthermore, the localization of the methylene blue dye is an example of a small molecule therapeutic that may be added to HA-based composition. The molecular weight of methylene blue is 319.85 g/mol.

### Administration of Therapeutic Cells

In some embodiments, a trail is created by first introducing a delivery needle into the spinal cord with a controlled path and rate of entry at a single injection site. Then, a trail of the therapeutic cells, and optionally therapeutic or diagnostic substances is deposited by a controlled retraction of the delivery needle coupled with ejection of the therapeutic cells, and optionally therapeutic or diagnostic substances through the needle. Some embodiments deliver a homogenous trail of therapeutic cells, and optionally therapeutic or diagnostic substances that may settle in aqueous solutions, such as cells, drug-loaded particles, or other solids. In such embodiments, the delivery medium may include a shear-thinning polymer or viscous liquid, such as hyaluronic acid.

A person of ordinary skill in the art will realize that the above description of embodiments and detailed examples below are given by way of example for illustrative purposes and that the invention may be embodied in other specific forms without departing from the spirit or essential characteristics of the disclosed and claimed inventions. The foregoing embodiments are therefore to be considered in all respects illustrative rather than limiting of the inventions described and claimed herein. This specification, including the Figures and examples and the data depicted in the Figures and examples provide a complete description of the inventions and the method of manufacturing and using the same sufficient for a person of ordinary skill in the art to practice the disclosed and claimed inventions throughout their entire scope. Various modifications or changes will be suggested to persons of ordinary skill in the art using no more than routine experimentation, and, many equivalents to the specific embodiments of the invention described herein may be possible with routine experimentation. Such equivalents are intended to within the spirit and purview of this application, as well as within the scope of the appended claims. Relative quantities of ingredients may be varied to optimize desired effects an additional ingredients may be added and/or other ingredients may be substituted for one or more ingredients described in this specification. Additional advantageous features and functionalities associated with the compositions, methods, and processes of the present invention will be apparent from the appended claims.

### Example 1: Operation of Experimental Injection Device in Surgical Setting

The present invention may is used to perform an experimental injection of neural stem cells into the spinal cord of pigs according to the following protocol. A portable, experimental injection device is constructed in accordance with the specification and figures of Provisional Application No. 62/384,505, filed on Sep. 7, 2016, which is incorporated by reference herein in its entirety. Yucatan mini-pigs of 20-25 kg are injected using a preferred embodiment of the invention described in Provisional Application No. 62/384,505 (“Experimental Injection Device”). Each pig receives a thoracic T10 laminectomy according to procedures well known in the art. No myelotomy is performed. The pia is nicked with a needle at the site of entry of the injection needle of the Experimental Injection Device. The injection needle utilized in the trial is composed of Nitinol® (nickel-titanium alloy), hereinafter referred to as “Nitinol needle.”

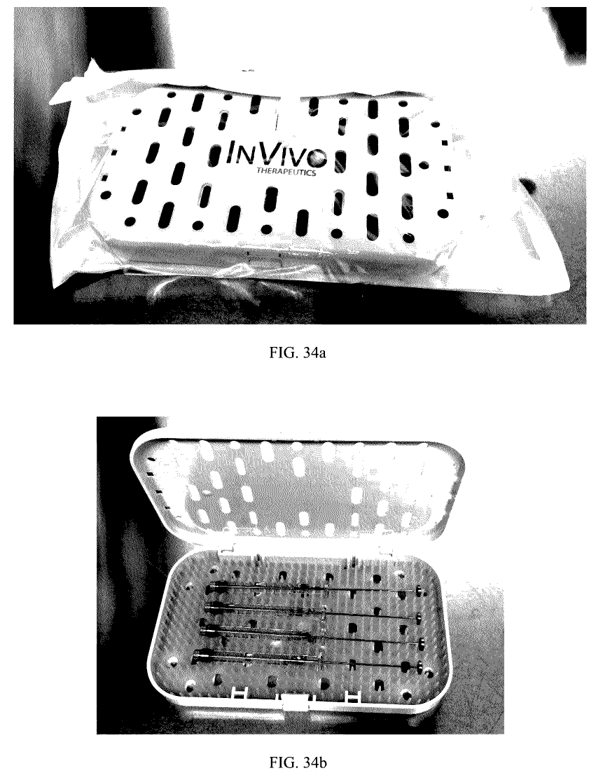

The injection utilizes an aqueous composition of hyaluronic acid 0.75% w/v in divalent ion-free phosphate buffered saline and human neural stem cells StemPro, ThermoFisher Scientific] at a concentration of 100,000 cells/μL. The composition is pre-filled into a syringe and transported to the surgical site in the manner shown in FIGS. 34a and 34b.

A 2 cm trail of cells in the spinal cord of each experimental mini-pig at a concentration of 100,000 cell/μL will be deposited, using the following administration parameters.

TABLE 1
Injection Administration Parameters

| | 2 cm Trail |
|---|---|
| Nitinol insertion & retraction rate | 0.5 |
| (mm/sec) | |
| Insertion - fluid delivery volume (μL/mm) | 0.07 |
| Retraction - fluid delivery volume (μL/mm) | 0.34 |
| Total injection volume (μL) | 8.2 |
| Total trail length (mm) | 20 |
| Total injection time (seconds) | 40 |

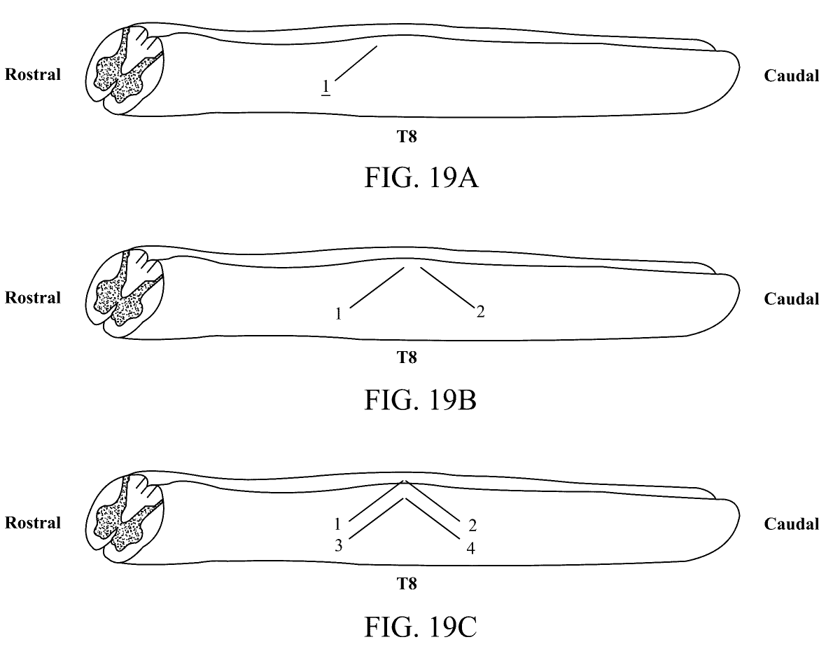

In the first experimental pig, a T10 laminectomy is performed according to conventional surgical procedures known in the art. A cell trail will be administered in the manner outlined in FIG. 19a. Trail 1: Stereotactic placement of a trail 1-2 mm right or left of midline and extending at ˜99 degrees for 2 cm (hypotenuse) in a caudal to rostral direction, ideally beginning (at maximal Nitinol extension) 2 mm above the most ventral aspect of the cord. Trail will begin in dorsal caudal white matter, travel rostrally and end in grey matter.

In the second experimental pig, a T10 laminectomy is performed according to conventional surgical procedures known in the art. A cell trail will be administered in the manner outlined in FIG. 19 b. Trail #1 and 2: Stereotactic placement of a trail 1-2 mm right or left of midline and extending at ˜99 degrees for 2 cm (hypotenuse) in a rostral to caudal direction, ideally beginning (at maximal Nitinol extension) 2 mm above the most ventral aspect of the cord. Trail will begin in dorsal caudal white matter, travel caudally and end in grey matter.

In the third experimental pig, a T10 laminectomy is performed according to conventional surgical procedures known in the art. A cell trail will be administered in the manner outlined in FIG. 19c. Trail #1 and 3: Stereotactic placement of a trail 1-2 mm right or left of midline and extending at ˜99 degrees for 2 cm (hypotenuse) in a caudal to rostral direction. Trail will begin in dorsal caudal white matter, travel rostrally and end in grey matter. Trail #2 and 4: Stereotactic placement of a trail ending 2 mm (along the hypotenuse) beneath the dorsal surface of the cord 1-2 mm right of midline and extending at ˜99 degrees for 2 cm (hypotenuse) in a rostral to caudal direction. Trail will begin in dorsal caudal white matter, travel caudally and end in grey matter.

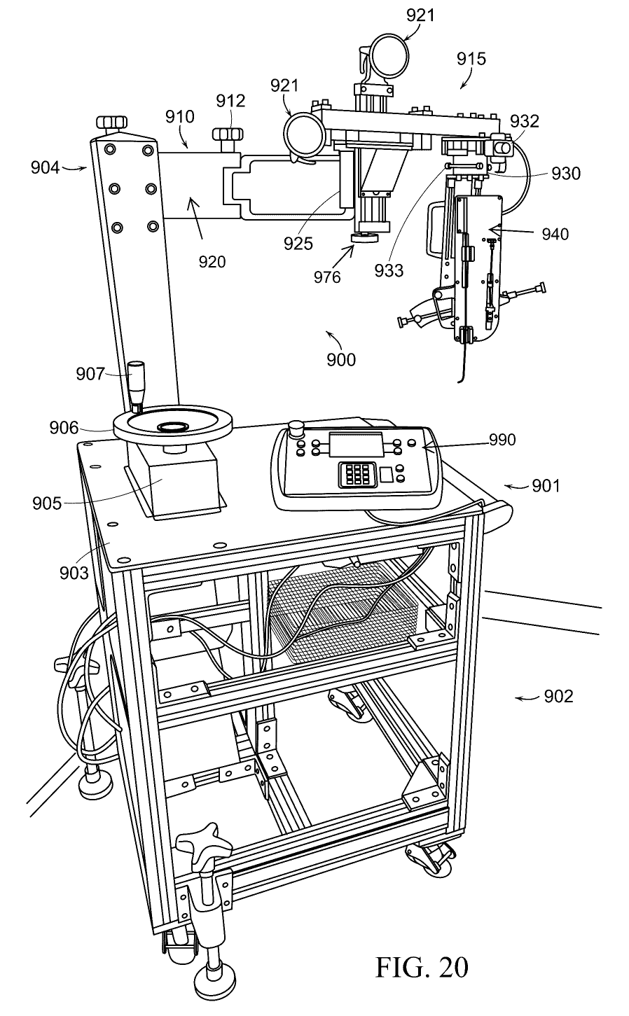

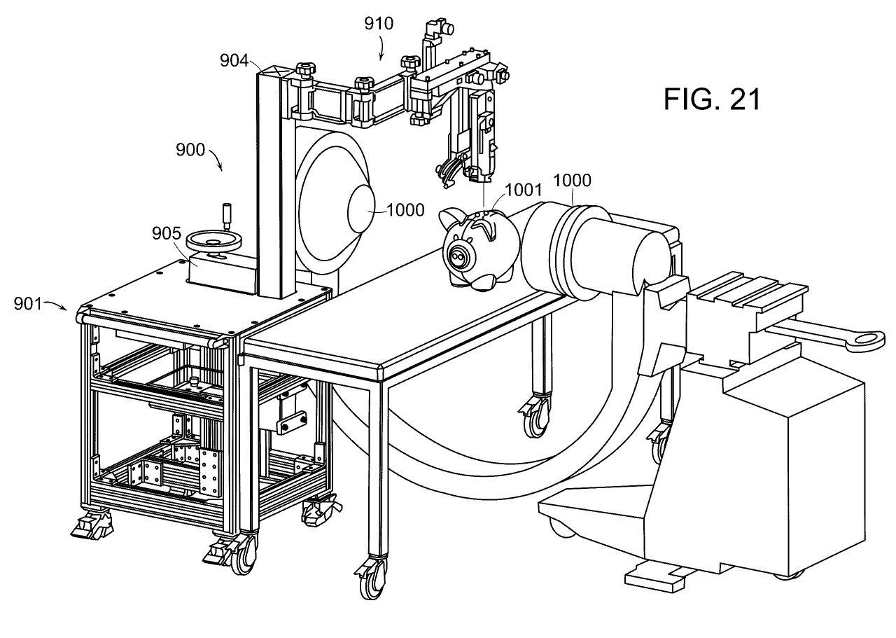

The administration of the human neural stem cells to the three experimental pigs will follow the following general procedure. A C-fluoroscope 1000 is positioned so as to allow lateral imaging of the cord by a radiologist. The Experimental Injection Device 900 of the type depicted in FIG. 20 mounted on portable cart is positioned next to operating table and checked for clearance by raising vertical macro height post 904 by manipulating macro height adjustment 905), and to determine the ability of positioning arm 910 to reach into the surgical\fluoroscope field, as graphically depicted in FIG. 21. Connection of the power source (not shown) and cable connection (not shown) of the motorized injection needle 960 to the motor box (not shown) is confirmed. The previously sterilized nitinol\guide needle assembly is flushed with sterile saline and checked for leaks. The experimental trail injection device 900 is powered-up by running the start-up procedure.

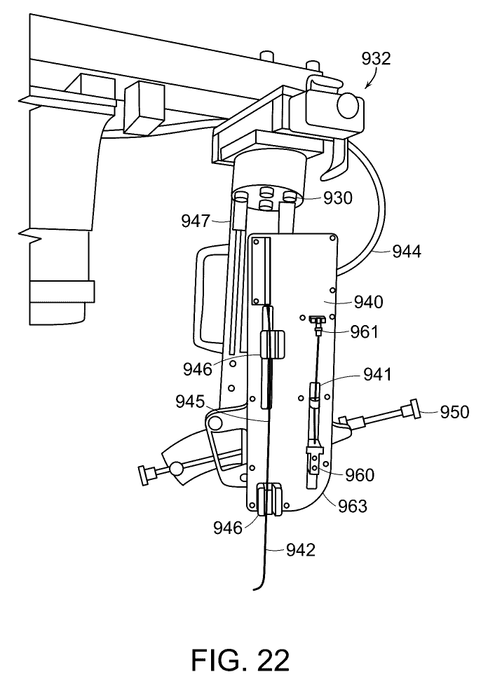

Next, the Nitinol injection needle 943 (not shown)\guide needle 942 assembly is secured to the motor assembly, as generally set forth in FIG. 22. A Hamilton syringe 941 is placed into the pump portion of motor syringe mechanism 960 and connected to the Nitinol needle. Infusion parameters are then programmed into control box 990 (See. FIG. 20). The dura of each experimental pig is tacked back by the surgeon according to conventional surgical procedures.

Thereafter, the Nitinol needle 943 is primed with the aqueous composition comprising hyaluronic acid and human neural stem cells. The stem cells may be StemPro® neural stem cells available from ThermoFisher Scientific. StemPro® Neural Stem Cells are derived from human fetal brain from qualified, traceable donors. The cells are isolated, cultured, and expanded under Good Manufacturing Practice (GMP) manufacturing standards in a California-licensed facility using a proprietary Reduced Oxygen Tension manufacturing process. Manufacture of cells in a reduced oxygen tension environment results in higher yields of highly potent immature stem cells compared to cells expanded in normal oxygen culture conditions. The suspension composition may be 0.75 wt. % hyaluronic acid in divalent ion-free PBS. The hyaluronic acid has a molecular weight of 1.1 to 1.8 MDa and may be obtained from LifeCore Biomedical, LLC

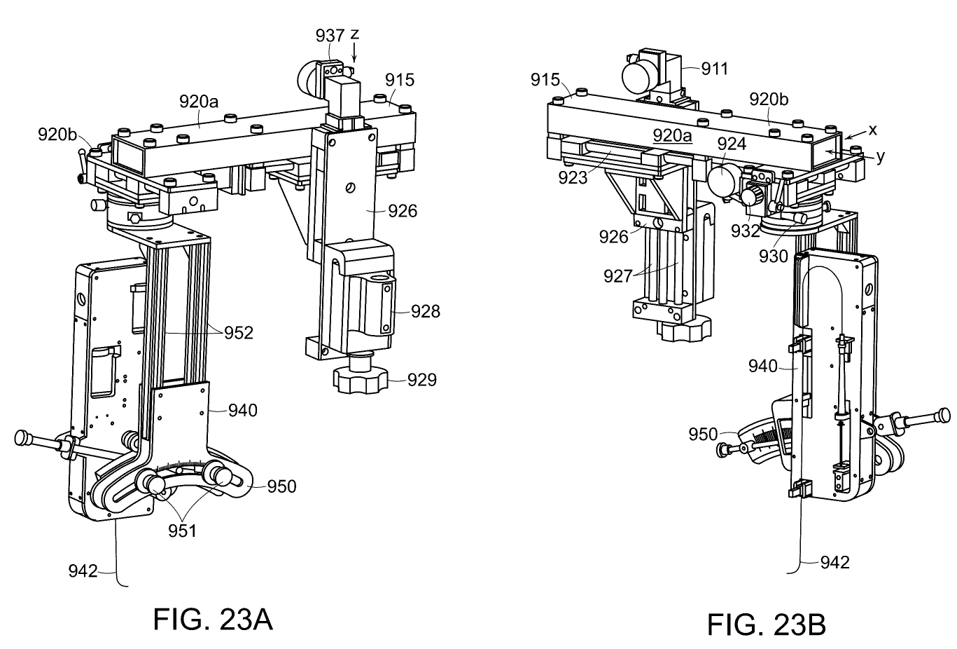

Wth reference generally to FIG. 21, the positioning arm 910 is used to localize the injection assembly over the pig 1001. The vertical post 904 is positioned so that the guide needle 943 is approximately 4 cm above the spinal cord of pig 1001. Using the micro-adjustment controls 911 and 931 (FIG. 23b), the guide needle 942 is lowered to about 1 mm right of midline and 1 cm above the dorsal aspect of the cord. Next, the Nitinol needle 943 is advanced and the macro\micro goniometer 950 is used to align the Nitinol needle 943 with the surface of the cord and parallel to the long axis of the cord. The fluoroscope 1000 is used to confirm alignment. The guide needle rotational (rotational micromanipulator) and angular positions (goniometer) are recorded.

Upon instruction by the neurosurgeon, the nitinol needle 943 is retracted. The micro goniometer 950 is then used to angle the guide needle 9 degrees into the spinal cord. An Anesthesiologist then hyperoxygenates the pig and then stops ventilation upon command by the Neurosurgeon. Time off the ventilator is recorded. Using the micro-adjustment controls 911 and 931 to lower the guide needle 942 the guide needle 942 is positioned to just slightly depress the pia. A small incision/entry hole (“nick”) may facilitate entry of the Nitinol needle 943 into the cord parenchyma. The Neurosurgeon then asks that fluoroscopy begins.

The Neurosurgeon calls for advancement of the Nitinol needle 943 under fluoroscopic guidance 1000. The Nitinol needle 943 is advanced to a fully extended position. A pre-flow of cells during nitinol advancement is set at 0.07 μL/mm. The fluid flow rate is then set to 0.34 μL/mm for retraction flow rate. See Table 1. Upon order of the Neurosurgeon the cell infusion and simultaneous Nitinol needle retraction is started. When the Nitinol needle 943 is fully retracted, the Neurosurgeon is informed, whereupon the Neurosurgeon raises the guide needle 942 away from the cord (at least 1-2 cm). Ventilation is then recommenced and the Neurosurgeon checks for retrograde leakage of the injection composition comprising human neural stem cells. The pia is then stitched to mark the location of the injection trail entrance.

### Example 2: In Vitro Therapeutic Trails Injection

FIGS. 18a and 18b illustrates trails injected at an angle in a “tent” formation around a prophetic injection site. Two opposing 2-cm long trails injected at 10 degree angles into a 0.6 wt. % agarose gel slab. The trails are composed of 0.75 wt. % hyaluronic acid in PBS and methylene blue was added for visualization purposes. FIG. 18a is a top view and 18b is a side view illustrating the angular injections and the described “tent” feature which may be used to inject a trail of cells and/or therapeutic substances proximal to an injury site in the spinal cord. Wth regard to the injection procedure, reference may be made to Example 1 above.

### Example 3: In Vitro Injection Angle Testing

An experimental test of the accuracy of injecting trails of cells and/or a therapeutic substance was conducted in an in vitro test model to determine the accuracy and extrusion depth of injections performed with an embodiment of the present invention. A certain embodiment of Experimental Injection Device 900 employing a goniometer 950 was utilized through the test procedure. Thus, a preliminary test of the accuracy of the goniometer angle mechanism was performed. The test was accomplished by measuring the extrusion depth at various goniometer angles.

Materials. Tests were performed utilizing gel slabs composed of 0.6 wt. % agarose in diH20. The liquid composition injected was a solution of 0.75 wt. % hyaluronic acid (“HA”) with methylene blue added for color. Trails of methylene blue were measured with a ruler.

Procedure. An injection needle 943 composed of nitinol was extruded 20 mm above the test gel slab. The goniometer on an embodiment of the device substantially similar to Embodiment 8 in Provisional Application No. 62/384,505 was used to angle the nitinol injection needle parallel to the surface of the gel. The injection assembly is described as an injection device for delivering a composition of cells and/or one or more therapeutic substance into the spinal cord of an animal, including a human, comprising: a) an injector device subassembly comprising: (1) an injection needle subassembly; (2) a separately provided prefilled syringe comprising an injection needle connector at one end and a plunger connected to a plunger rod; (3) at least one stepper motor; (4) one or more injector device subassembly mounting connectors; (5) an injection needle subassembly connector; (6) a first stepper motor connector between the injection needle subassembly and the stepper motor; and (7) a second stepper motor connector between the plunger rod and the stepper motor, wherein the second stepper motor connector is capable of controlling the volume and flow rate of the pre-filled syringe by actuation of the plunger rod in the operation of the injection device; b) a macro-positioning sub-assembly for roughly adjusting the orientation of the automated injector device sub-assembly along x, y and z axes relative to a prone animal or human positioned under the automated injection device, comprising a vertical height adjustable post, an adjustable articulated arm, and a micro-positioning subassembly; wherein the micro-positioning subassembly further comprises: a first horizontal support arm; a second horizontal support arm oriented at right angles to the first horizontal support arm; a rotatable stage member; and a goniometer comprising goniometer a macro-angular adjustment and a goniometer micro-angular adjustment; wherein the first horizontal support arm comprises one or more adjustable vertical support rail attached to a first vertical support rail micro-adjustor for adjusting the first horizontal support arm along the z axis; and further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more second adjustable goniometer rail attached at the top of the goniometer rail to the bottom surface of the rotatable stage; c) further comprising a separately provided injection needle subassembly, wherein the injection needle subassembly comprises: (i) a flexible metallic catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle; wherein the flexible metallic needle is dimensioned to pass through the telescoping two-part slide mechanism; further wherein the flexible wire catheter is rigidly secured to the interior of the outer cannula thereby providing for vertical movement of the outer cannula and attached flexible wire catheter upon actuation of the at least one stepper motor; and further wherein the flexible metallic catheter is capable of forming an injection needle service loop at the end of the catheter attached to the prefilled syringe; further wherein the outer cannula is attached to a first mounting block that connects to the at least one stepper motor connector between the injection needle subassembly and the at least one stepper motor; and wherein the inner cannula is rigidly attached to a second mounting block that rigidly connects to the injection needle subassembly connector of the injector device subassembly; and d) a programmable controller capable of controlling volume and flow rate of the pre-filled syringe in operation.

An angle of 8° was recorded. The nitinol injection needle was then retracted within the guide needle 942. The goniometer was adjusted to the desired angle of approach. With regard to the injection procedure reference may be made to Example 1 above for the general injection protocol. Further, the injection protocol generally followed the procedure outlined in Example 1. Controller 990 is used to control and display the position of the injection needle and to control and display (volume dispensed) the flow rate of composition of cells and/or therapeutic substance from the syringe, for example, a prefilled syringe in some embodiments. The skilled worker will understand how to program the programmable controller to perform the principal functions of advancing and retracting the needle and controlling the volume and flow rate of the contents of the injection syringe.

A pre-flow of the HA/methylene blue composition was set by the controller 990 at 0.07 μL/mm. The nitinol needle was then extruded 20 mm at 0.5 mm/sec into the agarose gel slab. The liquid composition of HA and methylene blue was flowed at a flow rate of 0.35 μL/mm upon retraction of the nitinol needle by setting the controller 990 to retract the nitinol injection needle. The methylene blue trails were measured with a ruler.

Testing Conditions. The following testing conditions for 20 mm trails were noted.

TABLE 2
Goniometer Angle Settings

| | | Goniometer angle |
|---|---|---|
| | Angle (°) (with | (~8 degrees as |
| Depth (mm) | respect to gel) | Parallel) |
| 2 | 5.7 | 2.3 |
| 4 | 11.5 | −3.5 |
| 6 | 17.5 | −9.5 |
| 8 | 23.6 | −14.5 |

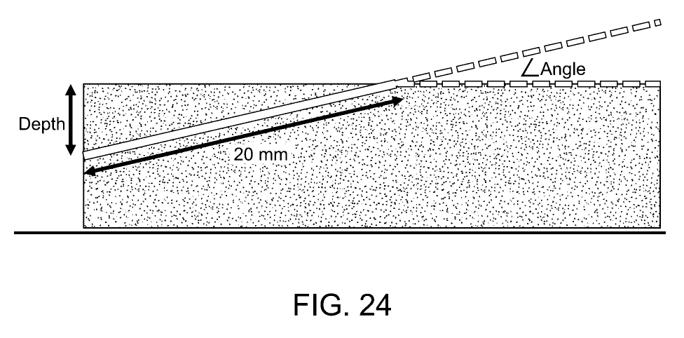

FIG. 24 depicts the angle measurements in accordance with the injection of a 20 mm trail of the liquid composition of HA and methylene blue in accordance with this Example 3.

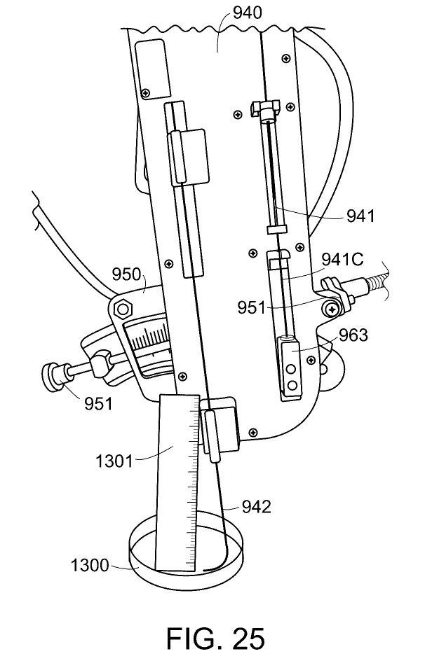

FIG. 25 depicts the testing setup for injection device 900 used in this Example 3. Reference can be made to FIG. 25. There is depicted an image of injector dispensing device 940 with guide needle 942 positioned over agarose gel slap 1300. As part of injector dispensing device 940, syringe 941 with plunger rod 941c positioned within motorized plunger drive 963 is depicted. Also, depicted is goniometer 950 and microadjustment knobs 951. Ruler 1301 is used to measure the trails of HA and methylene blue (not shown) in agarose gel slab 1300.

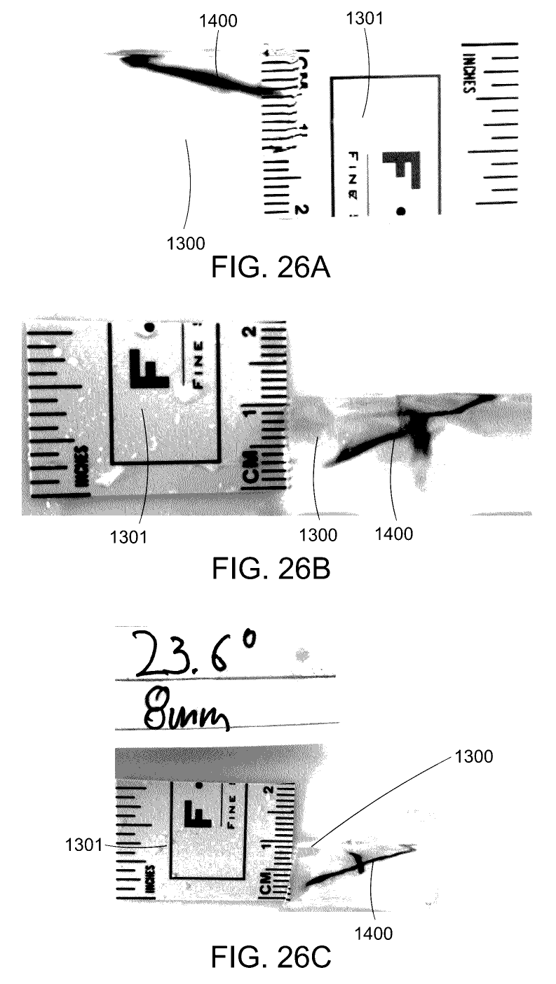

Results. The results obtained according to the foregoing in vitro test protocol are shown in FIGS. 26 a and 26b. FIGS. 26a and 26b show methylene blue trails 1400 in agarose slab 1300 injected at a 2 mm depth and a 5.7° angle. The result shown on ruler 1301 is 2-3 mm. FIGS. 26a, 26b and 26c shows the results of injections set at 4 mm, 6 mm and 8 mm, respectively. Methylene blue trails 1400 in agarose gel slabs 1300 as measured by ruler 1301 yielded the following results: (a) at a 4 mm depth setting and an injection angle of 11.5° the measured result was 4 mm; (b) at a 6 mm depth setting and an injection angle of 17.5° the measured result was 6-7 mm; and (c) at an 8 mm depth setting and an injection angle of 23.6° the measured result was 8 mm.

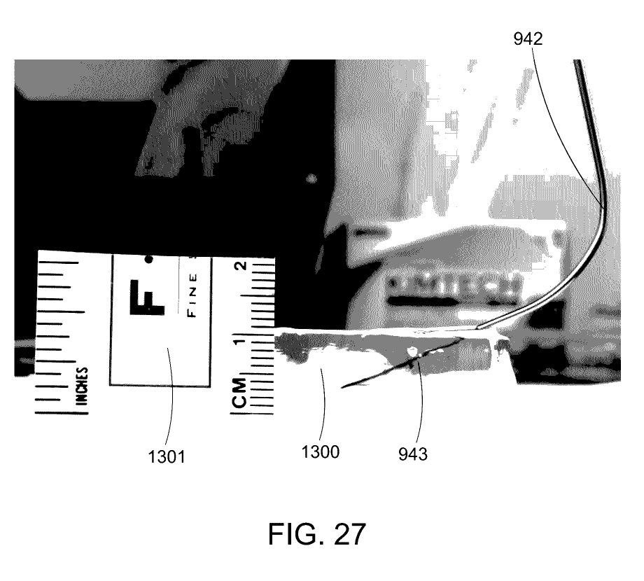

FIG. 27 is an image of a guide needle positioned at the surface of an agarose gel slab and an injection needle penetrating the agarose gel slab at a setting of 8 mm and an injection angle of 23.6° yielding a trail of 8 mm in accordance with Example 3.

### Example 4: Effect of Neural Stem Cell Addition on the Mechanical Properties (Storage & Loss Modulus) of the Injectable Medium

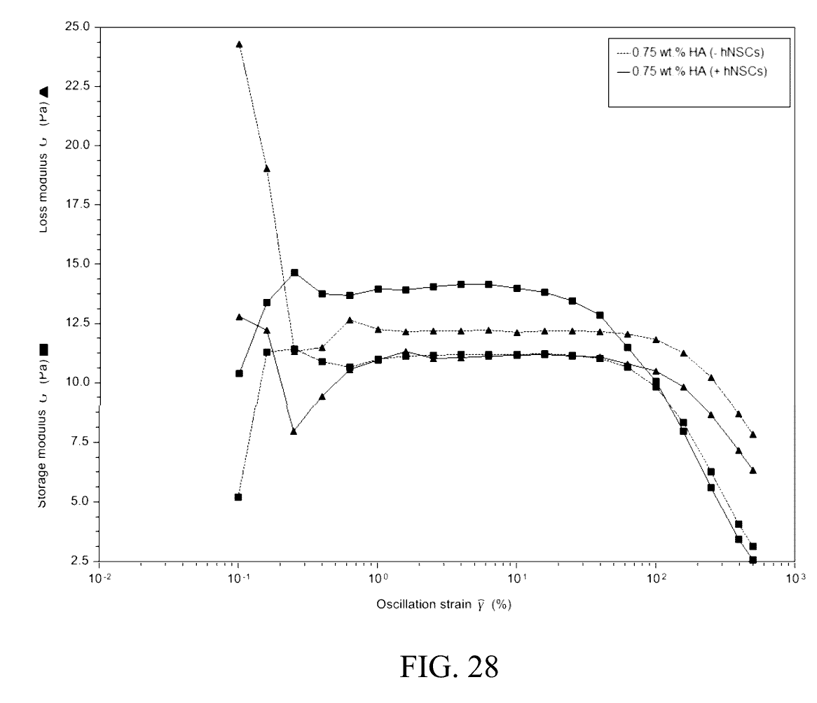

FIG. 28 depicts a rheology plot showing the effect of human neural stem cell addition on the mechanical properties (storage & loss modulus) of the composition. The cell containing HA composition was prepared as described (pelleted 100,000 hNSCs/μL of HA, disrupted pellet with PBS, added 1 wt. % HA to achieve a final HA concentration of 0.75 wt. %). The HA composition without cells was prepared in the same manner. Testing was performed on a TA Ares G2 rheometer fitted with a 25 mm parallel plate fixture (0.5 mm gap). Testing conditions: Strain sweep @ 20° C. from 0.1 to 500% strain, 1 Hz. Within the linear viscoelastic region (1% strain), the storage and loss moduli of 0.75 wt. % HA without cells were both approximately 10 Pa. The storage and loss moduli overlapped or the storage modulus was higher than loss modulus, indicating a more fluid-like composition. With the addition of cells, the storage modulus of the composition increased to ˜14 Pa and the loss modulus remained at ˜10 Pa. Notably, the storage modulus of the HA-cell mixture was higher than the loss modulus, indicating that the addition of cells resulted in the formation of a gel. This final gel-like composition may enable the long-term suspension of cells (for storage\transport) and the homogenous delivery of cells to sites of therapeutic interest.

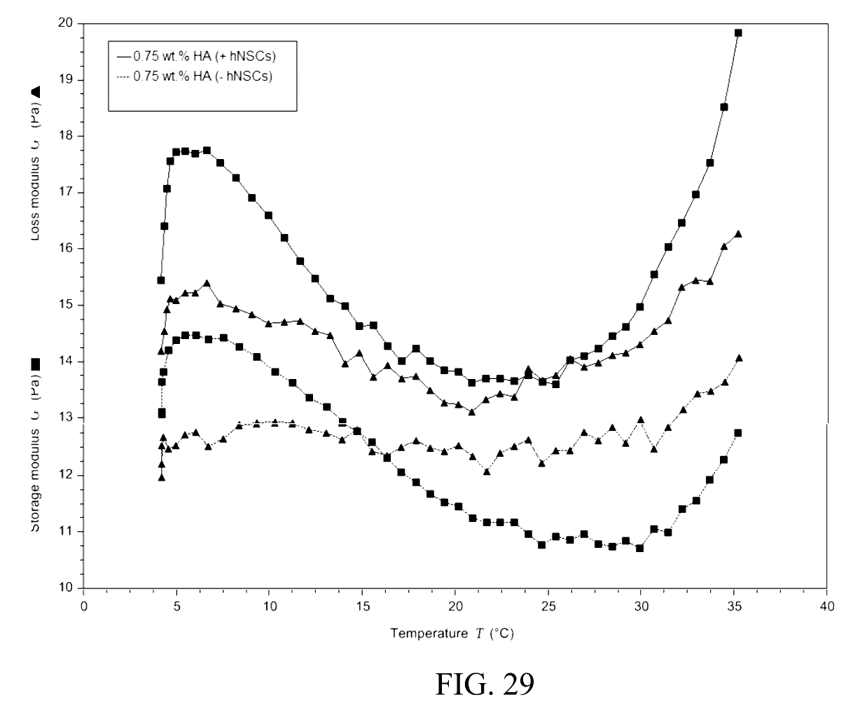

FIG. 29 depicts a rheology plot showing the effect of human neural stem cell addition on the temperature-dependent properties (storage & loss modulus) of the composition. The cell containing HA composition was prepared as described (pelleted 100,000 hNSCs/μL of HA, disrupted pellet with PBS, added 1 wt. % HA to achieve a final HA concentration of 0.75 wt. %). The HA composition without cells was prepared in the same manner. Testing was performed on a TA Ares G2 rheometer fitted with a 25 mm parallel plate fixture (0.5 mm gap). Testing conditions: Temperature ramp from 4° C. to 37° C. (1% strain, 1 Hz). Similar to FIG. 28, with the addition of cells, the storage modulus of the HA-cell mixture was higher than the loss modulus, indicating that the addition of cells resulted in the formation of a gel. This final gel-like composition may enable the long-term suspension of cells (for storage\transport) and the homogenous delivery of cells to sites of therapeutic interest.

The temperature sweep shows a reduction in storage modulus from 4° C. (storage conditions) to 20° C. and 37° C. (use conditions). The elevated storage modulus (˜18 Pa for the cell containing composition) at 4° C. enables the maintained of cell suspension during storage and transport of the refrigerated cell suspension. Reduction in storage modulus at the use temperature (˜14 Pa, 20° C.) may result in less shear on the cells during injection, and potentially increased cell viability compared to injection of more viscous suspensions. The increased storage modulus after ˜27° C. may be due to sample evaporation in the testing fixture.

### Example 5: Testing of Needle Speed Range; Range of 0.1 to 5 mm/sec

The needle speed of an embodiment of the injection device for delivering trails of cells and/or a therapeutic substance was testing according to the following method.

Method. Retract the needle until approximately 2-5 mm is showing beyond the tip of the guide needle. Zero the position readout on the display. Select the speeds 0.1 mm/sec, 1.5 mm/sec and 5 mm/sec one at a time. Advance the needle at the given speed for the specified time. Measure the change in needle protrusion and compare with the theoretical value. Confirm the distance reading on the screen and record. Measuring equipment used was Calipers-Mitutoyo Digital.

Results.

TABLE 3
Needle Speed Results

| | Test | Ex- | Start | End | Distance | |
|---|---|---|---|---|---|---|
| Speed | time | pected | Length | Length | Advanced | Displayed |
| (mm/s) | (s) | length | (mm) | (mm) | (mm) | Length |
| 0.1 | 120 s | 12 mm | 3.79 | 15.07 | 11.28 | 11.98 |
| 1.5 | 20 s | 30 mm | 2.88 | 33.28 | 30.4 | 30.32 |
| 5 | 10 s | 50 mm | 2.84 | 53.76 | 50.92 | 51.59 |
| 10 | 4 s | 40 mm | 3.93 | 46.16 | 42.23 | 42.23 |

Based on the testing performed, the system display is accurate to within about 0.7 mm. A significant portion of this error is related to the measurement method. The distance advanced is different from the expected value largely due to the reaction time for starting and stopping the system at the appropriate time.

### Example 5. Relative Fluid Delivery Range; Relative Fluid Delivery Range 0.01-8 μL/mm. Absolute Fluid Delivery Range 0.01-25 μL/sec

Method. Testing of distances traveled by the injection needle and the amount of fluid dispensed were measure according to the following method.

Method. A syringe was filled with water and a needle assembly was attached. The syringe was primed to remove air from the injection system. The needle was then advanced until approximately 50-55 mm of the needle tip was showing beyond the tip of the guide needle. The position was zeroed in the controller display. Next, the needle speed was selected and the indicated fluid rate in FIG. 28 [formerly] 57 was set. The controller was set to dispense liquid. Thereafter, the needle was retracted at the indicated speed for the time specified in FIG. 28 [formerly 57]. The dispensed water was collected in a sample tray. The needle protrusion was measured and compared to the theoretical value. The distance reading on displayed on the controller was recorded. The liquid was then weighed and the measurement was recorded in Table 4 below. Equipment utilized included Calipers-Mitutoyo digital and a Mettler-Toledo balance XS-205.

Results. The results are reported in FIG. 28 [formerly 57] and below. Calculated values included the following:

Abs. Fluid Rate—The fluid delivery rate in μl/s. Calculated by multiplying Needle speed (mm/s) by Fluid Rate (μl/mm).

Expected Needle Travel—Calculated by multiplying Needle Speed (mm/s) by Test time (s).

Expected Total Dispense—Calculated by multiplying Fluid Rate (μl/mm) by Expected Needle Travel (mm).

Actual Syringe Travel—Calculated by subtracting the Syringe End Volume (μl) from the Syringe Start Volume (μl). Actual Needle Travel—Calculated by subtracting the Needle Start length (mm) from the Needle End length (mm).

Actual Fluid Rate by Distance—Calculated by dividing the Actual Syringe Travel (μl) by the Actual Needle Travel (mm).

The absolute distances travelled and amount of fluid varied from the expected values in the following manner. Faster needle speeds with shorter test times exhibited poorer results. This is largely due to reaction time in starting and stopping the test which has a greater effect over shorter test times.

The weighed dispensed fluid values were generally close to the fluid dispensed by distance (reading syringe graduations), but consistently lower. This can be explained by evaporation, air dissolved in solution and small amounts of water clinging to the needle after dispense. An effort was made to get the water off of the tip of the syringe, but it was difficult to confirm this

Maximum dispense rate error observed is 6.5%. Maximum needle speed error observed was 17.8% (1.6 mm out of 9 mm expected). This was observed on a 3 second test at 3 mm/sec. If reaction time accounted for 0.5 seconds of error, the distance error would have been 1.5 mm. Needle distance measurement error is estimated at about 0.5 mm. Longer tests showed distance errors of 2% maximum. The foregoing results demonstrate that expected needle travel, expected total; fluid dispensed and expected absolute fluid rate are well within expected and acceptable tolerances as demonstrated by the values actually obtained in Example 5 and reported in FIG. 27.

### Example 6. Long Term Survival and Differentiation of Neural Stem Cells Delivered in the HA Carrier

Successful implementation of cellular therapy requires cell survival, differentiation (in the case of stem cells), and implanted cell integration with tissue. Human neural stem cells in the HA composition have been extensively studied in uninjured nude rats and in nude rat models of spinal cord injury. For delivery of human neural stem cells trails in nude rat spinal cord a T10 to T11 laminectomy was performed in a nude rat. StemPro® Neural Stem Cells were combined with 0.75 wt. % HA at a concentration of 100,000 cells/uL and loaded into a 100 uL Hamilton syringe. The syringe was secured to the injection apparatus and the 29G nitinol injection needle was primed with cells. The guide needle was lowered to the exposed surface of the rat spinal cord, the dura was cut with a 26G needle to facilitate entry of the injection needle, and the nitinol was extruded 12 mm at an angle of 9 degrees into the rat cord. Upon full extension, flow of cell suspension was initiated (10 uL/min) along with needle retraction (0.5 mm/second). Following injection, the overlaying muscle and skin as closed and the animal was allowed to recover. FIG. 30 shows successful creation of a trail and survival of human neural stem cells in the nude rat spinal cord after one month. The cells were labeled for human cytoplasm (STEM121) and doublecortin (DCX) markers, showing cell survival and neuronal precursor differentiation. Furthermore, cells were observed migrating along the white matter tracts, showing that the HA carrier allows for cells to integrate into the surrounding tissue environment rather than being constrained to the injection site by the biomaterial.

Longer term survival was evaluated in a nude rat by performing a laminectomy at T8, lowering a bent 30G needle, and injecting a 4 mm trail of human neural stem cells in the cranial direction. After three months the rat was perfused and the spinal cord was cryosectioned for histology. The sections were labeled for makers of human cytoplasm (STEM121), human astrocytes (STEM123), and axons (TUJ1). FIG. 32 shows survival (STEM121) and differentiation (STEM 123 & TUJ1) of the human neural stem cells after 3 months.

To test cell survival when delivered in the HA carrier into an injured environment, a 200 kDyne contusion was induced in a nude rat at T8 with a IH Impactor (Infinite Horizons, Percision Systems and Instrumentation LLC). Two weeks later, a T10 to T11 laminectomy was performed to allow positioning of the guide needle. StemPro® Neural Stem Cells were combined with 0.75 wt. % HA (1.01-1.8 MDa, LifeCore Biomedical) at a concentration of 100,000 cells/μL and loaded into a 100 μL Hamilton syringe. The syringe was secured to the injection apparatus and the 29G nitinol injection needle was primed with cells. The guide needle was lowered to the exposed surface of the rat spinal cord, the dura was cut with a 26G needle to facilitate entry of the injection needle, and the nitinol was extruded 12 mm at an angle of 9 degrees into the rat cord. Upon full extension, flow of cell suspension was initiated (10 μL/min) along with needle retraction (0.5 mm/second). Following injection, the overlaying muscle and skin as closed and the animal was allowed to recover. The rat was perfused after three months and the spinal cord was explanted for histology. FIG. 32 shows immunohistochemical staining for human cytoplasm (STEM121) and DAPI showing survival of human neural stem cells delivered in a trail through the contusion injury in a nude rat. This demonstrates that cell trails can survive and transverse injuries in the spinal cord.

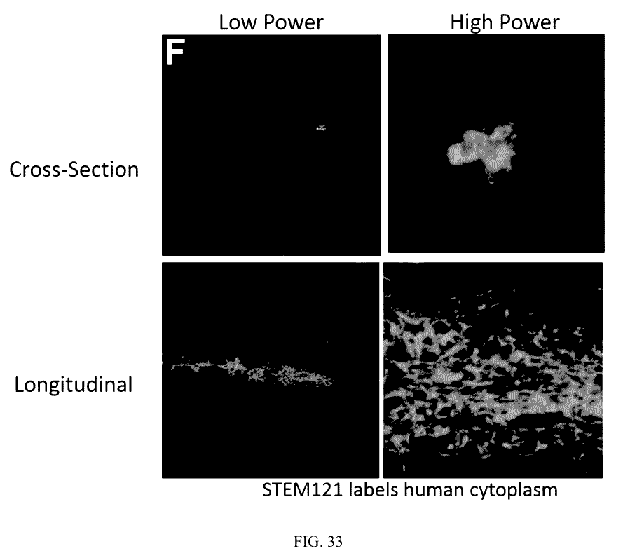

The ability of the HA composition to enable transportation and post-injection cell survival was also evaluated in a porcine model. The human neural stem cells-HA composition (100,000 cells/μL in 0.75 wt. % HA; 1.01-1.8 MDa MW, LifeCore Biomedical) was pre-filled into 100 μL Hamilton syringes and shipped overnight at 4° C. to the surgical site. The cell suspension was subsequently injected as a trail into the spinal cord of an immunosuppressed Gottingen minipig. As shown in FIG. 33, the cells survived, extended processes, and formed a neural plexus after one week. Again this shows the suitability of the HA formulation for cell storage, transport, and viable cell injection.

### Gene Therapy

Gene therapy as used herein refers to the transfer of genetic material (e.g., DNA or RNA) of interest into a subject to treat or prevent a genetic or acquired disease or condition. The genetic material of interest encodes a product (e.g., a protein, polypeptide, and peptide, functional RNA, antisense) whose in vivo production is desired. For example, the genetic material of interest encodes a hormone, receptor, enzyme polypeptide or peptide of therapeutic value. For a review see “Gene Therapy” in Advances in Pharmacology, Academic Press, San Diego, Calif., 1997.

Somatic gene therapy is, i.e. gene therapy to non-reproductive cells, is divided into two basis types of therapy. Ex vivo gene therapy involves modification of cells outside the body, which are then transplanted back into the body. The cells are exposed to a virus that contains the desired gene of interest. The virus enters the cells and introduces the gene into the targeted cells' DNA upon transplantation back into the body. In vivo gene therapy involves transfer of a gene into the subject's cells inside of the subject's body.

Various viral vectors are known in the art, which include retroviruses or adenoviruses. Other viral vectors known in the art are adeno-associated viral vectors, lentiviruses, pox viruses, alphaviruses and herpes viruses.

Administration of gene transfer may be performed through, for example, injection by spinal tap into the cerebrospinal fluid surrounding the brain and spinal cord. The gene may be delivered in a modified virus that carries the genes to cells in the subject's body. An example would be the clinical trial described as “Intrathecal Administration of scAAV9/JeT-GAN for the Treatment of Giant Axonal Neuropathy,” as Trial No. NCT02362438 at www.Clinical Trials.gov.

Alternatively, the gene of interest may be transferred by infusion following a surgical procedure to infuse the viral vector and gene into the brain of a subject. An example would be to treat Parkinson's disease, such as the clinical trial described as “Phase 1 Open-Label Dose Escalation Safety Study of Convection Enhanced Delivery (CED) of Adeno-Associated Virus Encoding Glial Cell Line-Derived Neurotrophic Factor (AAV2-GDNF) in Subjects with Advanced Parkinson's Disease,” identified as Study NCT01611581 at www.ClinicalTrials.gov. See also, Björklund A, Kirik D, Rosenblad C, Georgievska B, Lundberg C, Mandel R J. Towards a neuroprotective gene therapy for Parkinson's disease: use of adenovirus, AAV and lentivirus vectors for gene transfer of GDNF to the nigrostriatal system in the rat Parkinson model. Brain Res. 2000 Dec. 15; 886(1-2):82-98. Review.

Another exemplary trial is NCT01973543, entitled “An Open-label Safety and Efficacy Study of VY-AADC01 Administered by MRI-Guided Convective Infusion Into the Putamen of Subjects Wth Parkinson's Disease with Fluctuating Responses to Levodopa. In the latter study, a hAADC gene is packaged into a gene transfer vector derived from a common, non-pathogenic virus (AAV2) to which >90% of humans have been exposed. The investigational drug, termed VY-AADC01, will be injected directly into the striatum during a neurosurgical procedure that is performed with real-time MRI imaging to monitor delivery.

Additional references Experimental Eye Research 89; 301-310. Bible E, Chau, Y S, Alexander M R, Price J, Shakesheff K R, Modo M. (2009) The support of neural stem cells transplanted into stroke-induced brain cavities by PLGA particles. Various publications, including patents, published applications, technical articles and scholarly articles are cited throughout the specification. Each of these cited publications is incorporated by reference herein, in its entirety.

What is claimed is:

1. An injectable medium comprising therapeutic cells, and optionally therapeutic or diagnostic substances, suitable for injection into an anatomical space of a human or animal subject, comprising:
(a) therapeutic cells, and optionally therapeutic or diagnostic substances;
(b) a pharmaceutically acceptable diluent comprising hyaluronic acid;
wherein the injectable medium has a storage modulus within the range of 5-25 Pa.

2. The injectable medium according to claim 1, wherein the hyaluronic acid is formulated at a concentration of about 0.5 wt. % to about 1 wt. % in the injectable medium; and further wherein the hyaluronic acid has a molecular weight of about 700 kDa to about 1,900 kDa.

3. An injectable medium comprising neural stem cells, and optionally therapeutic or diagnostic substances, suitable for injection into an anatomical space of a human or animal subject, comprising:
(a) human neural stem cells, and optionally therapeutic or diagnostic substances;
(b) a pharmaceutically acceptable diluent comprising hyaluronic acid;
wherein injectable medium has a storage modulus within the range of 5-25 Pa.

4. The injectable medium according to claim 3, wherein the hyaluronic acid is formulated at a concentration of about 0.5 wt. % to about 1 wt. % in the injectable medium; and further wherein the hyaluronic acid has a molecular weight of about 700 kDa to about 1,900 kDa.

5. The injectable medium according to claim 1, wherein the therapeutic cells are selected from the group consisting of neural stem cells, pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem cells, genetically modified cells, and the differentiated progeny of any of the above.

6. The injectable medium of claim 3, wherein the neural stem cells are undifferentiated progeny of human neural stem cells.

7. The injectable medium according to claim 3, wherein the neural stem cells are differentiated progeny of human neural stem cells.

8. The injectable medium according to claim 1, wherein the therapeutic cells are obtained by the further step of centrifuging the cells in vitro to obtain a pellet of spheres, aggregates or single cells.

9. The injectable medium according to claim 3, wherein the human neural stem cells are obtained by the further step of centrifuging the cells in vitro to obtain a pellet of spheres, aggregates or single cells.

10. The injectable medium according to claim 1, wherein the pharmaceutically acceptable diluent is selected from the group consisting of: divalent ion-free buffed salt solution; phosphate buffered saline; cell culture medium, isotonic saline, hanks buffered salt solution, HEPES buffered salt solution, and artificial cerebrospinal fluid.

11. The injectable medium according to claim 3, wherein the pharmaceutically acceptable diluent is selected from the group consisting of: divalent ion-free buffed salt solution; phosphate buffered saline; cell culture medium, isotonic saline, hanks buffered salt solution, HEPES buffered salt solution, and artificial cerebrospinal fluid.

12. The injectable medium according to claim 1, wherein the pharmaceutically acceptable buffer further comprises ascorbic acid, glucose, or glutamine.

13. The injectable medium according to claim 3, wherein the pharmaceutically acceptable buffer further comprises ascorbic acid, glucose, or glutamine.

14. The injectable medium according to claim 1, wherein the pharmaceutically acceptable buffer further comprises a neuroprotective, angiogenic, anti-angiogenic or neuroregenerative pharmaceutical substance.

15. The injectable medium according to claim 3, wherein the pharmaceutically acceptable buffer further comprises a neuroprotective, angiogenic, anti-angiogenic or neuroregenerative pharmaceutical substance.

16. The injectable medium according to claim 1, wherein the pharmaceutically acceptable buffer further comprises at least one factor capable of stimulating endogenous stem cells.

17. The injectable medium according to claim 3, wherein the pharmaceutically acceptable buffer further comprises at least one factor capable of stimulating endogenous stem cells.

18. The injectable medium according to claim 1, wherein the pharmaceutically acceptable buffer further comprises a drug and/or growth factor selected from the group consisting of: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

19. The injectable medium according to claim 3, wherein the pharmaceutically acceptable buffer further comprises a drug and/or growth factor selected from the group consisting of: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

20. The injectable medium according to claim 1, wherein the composition enables the therapeutic cells to be suspended uniformly for up to five days.

21. The injectable medium according to claim 3, wherein the composition enables the therapeutic cells to be suspended uniformly for up to five days.

22. The injectable medium according to claim 1, wherein the anatomical space is a human brain.

23. The injectable medium according to claim 1, wherein the anatomical space is a human spinal cord.

24. A method of preparing an injectable medium comprising therapeutic cells, and optionally one or more therapeutic or diagnostic substance, suitable for injection into an anatomical space of a human or animal subject, comprising the steps of:
(a) introducing into a sterilized vial a desired quantity of therapeutic cells, and optionally one or more therapeutic or diagnostic substance;
(b) adding to the vial a pharmaceutically acceptable diluent comprising hyaluronic acid;
(c) mixing the above injectable medium until a substantially uniform suspension is obtained having a storage modulus within the range of 5-25 Pa.

25. The method according to claim 24, wherein the hyaluronic acid is formulated at a concentration of about 0.5 wt. % to about 1 wt. % in the injectable medium; and further wherein the hyaluronic acid has a molecular weight of about 700 kDa to about 1,900 kDa.

26. A method of preparing an injectable medium comprising neural stem cells, and optionally therapeutic or diagnostic substances suitable for injection into an anatomical space of a human or animal subject, comprising the steps of:
(a) introducing into a sterilized vial a desired quantity of human neural stem cells;
(b) adding to the vial a pharmaceutically acceptable diluent comprising hyaluronic acid;
(c) mixing the above injectable medium until a substantially uniform suspension is obtained having a storage modulus within the range of 5-25 Pa.

27. The method according to claim 26, wherein the hyaluronic acid is formulated at a concentration of about 0.5 wt. % to about 1 wt. % in the injectable medium; and further wherein the hyaluronic acid has a molecular weight of about 700 kDa to about 1,900 kDa.

28. The method according to claim 24, wherein the therapeutic cells are selected from the group consisting of neural stem cells, pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem cells, genetically modified cells, and the differentiated progeny of any of the above,

29. The method of claim 26, wherein the neural stem cells are undifferentiated progeny of human neural stem cells.

30. The method of claim 26, wherein the neural stem cells are differentiated progeny of human neural stem cells.

31. The method according to claim 24, wherein the therapeutic cells are obtained by the further step of centrifuging the cells in vitro to obtain a pellet of spheres, aggregates or single cells.

32. The method according to claim 26, wherein the human neural stem cells are obtained by the further step of centrifuging the cells in vitro to obtain a pellet of spheres, aggregates or single cells.

33. The method according to claim 24, wherein the pharmaceutically acceptable diluent is selected from the group consisting of: divalent ion-free buffed salt solution; phosphate buffered saline; cell culture medium, isotonic saline, hanks buffered salt solution, HEPES buffered salt solution, and artificial cerebrospinal fluid.

34. The method according to claim 26, wherein the pharmaceutically acceptable diluent is selected from the group consisting of: divalent ion-free buffed salt solution; phosphate buffered saline; cell culture medium, isotonic saline, hanks buffered salt solution, HEPES buffered salt solution, and artificial cerebrospinal fluid.

35. The method according to claim 24, wherein the pharmaceutically acceptable buffer further comprises ascorbic acid, glucose, or glutamine.

36. The method according to claim 26, wherein the pharmaceutically acceptable buffer further comprises ascorbic acid, glucose, or glutamine.

37. The method according to claim 24, wherein the pharmaceutically acceptable buffer further comprises a neuroprotective, angiogenic, anti-angiogenic or neuroregenerative pharmaceutical substance.

38. The method according to claim 26, wherein the pharmaceutically acceptable buffer further comprises a neuroprotective, angiogenic, anti-angiogenic or neuroregenerative pharmaceutical substance.

39. The method according to claim 24, wherein the pharmaceutically acceptable buffer further comprises at least one factor capable of stimulating endogenous stem cells.

40. The method according to claim 26, wherein the pharmaceutically acceptable buffer further comprises at least one factor capable of stimulating endogenous stem cells.

41. The method according to claim 24, wherein the pharmaceutically acceptable buffer further comprises a drug and/or growth factor selected from the group consisting of: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

42. The method according to claim 26, wherein the pharmaceutically acceptable buffer further comprises a drug and/or growth factor selected from the group consisting of: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

43. The method according to claim 24, wherein the injectable medium enables the therapeutic cells to be suspended uniformly for up to five days.

44. The method according to claim 26, wherein the injectable medium enables the human neural stem cells to be suspended uniformly for up to five days.

45. The method according to claim 24, wherein the anatomical space is a human brain.

46. The method according to claim 24, wherein the anatomical space is a human spinal cord.

47. The composition according to claim 1, wherein the concentration of therapeutic cells is about 1×10⁴ to about 1×10⁸ cells per milliliter of injectable medium.

48. The composition according to claim 3, wherein the concentration of human neural stem cells is about 1×10⁴ to about 1×10⁸ cells per milliliter of injectable medium.

49. The method according to claim 24, wherein the concentration of therapeutic cells is about 1×10⁴ to about 1×10⁸ cells per milliliter of injectable medium.

50. The method according to claim 26, wherein the concentration of human neural stem cells is about 1×10⁴ to about 1×10⁸ cells per milliliter of injectable medium.

51. The method according to claim 24, wherein step (d) is performed using a dual-asymmetric centrifugal mixer

52. The method according to claim 26, wherein step (d) is performed using a dual-asymmetric centrifugal mixer
